(19) United States
(12) Patent Application Publication
(10) Pub. No.: US 2017/0273906 A1
(43) Pub. Date: Sep. 28, 2017

(54) TARGETED LIPOSOMES ENCAPSULATING IRON COMPLEXES AND THEIR USES

(71) Applicants: Ramot at Tel-Aviv University Ltd., Tel Aviv (IL); Tel Hashomer Medical Research Infrastructure and Services Ltd., Ramat Gan (IL)

(72) Inventors: Jonathan LEOR, Hadera (IL); Tamar BEN-MORDECHAI, Rishon LeZion (IL); Radka HOLBOVA-GELKOFF, Prerov (CZ); Olga YARENCHYK, Nitzaney Oz (IL); Rimona MARGALIT, Givataim (IL); Inbar ELRON-GROSS, Ramat Hasharon (IL); Yifat GLUCKSAM-GALNOY, Kfar-Saba (IL)

(73) Assignees: Ramot at Tel-Aviv University Ltd., Tel Aviv (IL); Tel Hashomer Medical Research Infrastructure and S ervices Ltd., Ramat Gan (IL)

(21) Appl. No.: 15/505,464

(22) PCT Filed: Jun. 18, 2015
(86) PCT No.: PCT/IL2015/050624

§ 371 Date: Feb. 21, 2017

Related U.S. Application Data
(60) Provisional application No. 62/040,206, filed on Aug. 21, 2014.

(51) Int. Cl. A61K 9/127; A61K 31/555

(52) U.S. Cl. CPC A61K 9/1271; A61K 31/555

(57) ABSTRACT

Provided are liposomes including a lipid membrane enclosing an intraliposomal compartment. The liposomes may encapsulate an iron complex, including an iron moiety and a macromolecule, in the intraliposomal compartment. The liposomes may additionally have a targeting moiety associated with the liposomal membranes. The targeting moiety may be exposed at the liposome's outer surface. The liposomes exhibit an immunomodulatory effect or anti-cancer effect depending on the molar ratio between the lipids forming the lipid membrane and the iron complex. As such, the liposomes can be used to treat types of inflammation, particularly inflammations that are not associated or caused by infection or be used to treat cancer. Further provided are compositions including the disclosed liposomes, methods of using and kits including the liposomes.

### TECHNOLOGICAL FIELD

This invention relates to liposomal formulations comprising iron complexes.

### BACKGROUND ART

References considered to be relevant as background to the presently disclosed subject matter are listed below:
US patent application publication No. US2005/0008687
Japanese patent publication No. JP58213711
European patent publication No. EP0066884
International patent application publication No. WO11128896

Acknowledgement of the above references herein is not to be inferred as meaning that these are in any way relevant to the patentability of the presently disclosed subject matter.

### BACKGROUND

Liposomes encapsulating metal complexes were previously described, for example, by JP58213711 and EP0066884 describe liposome including iron porphyrin complex.

In addition, US2005/0008687 describes metalloporphyrin-complex-embedded liposomes and their use as anticancer agents or antioxidants.

Further, WO11128896 describes iron oxide nanoparticles for use in the treatment of non-infectious inflammatory disorders.

### GENERAL DESCRIPTION

The present disclosure provides, in accordance with a first of its aspects, a liposome comprising a lipid membrane enclosing an intraliposomal compartment, said liposome: (i) encapsulates in the intraliposomal compartment an iron complex comprising an iron moiety and a macromolecule; and (ii) has associated to its membrane a targeting moiety, the targeting moiety being exposed at the liposome's outer surface.

In accordance with a second aspect, the present disclosure provides a therapeutic method comprising administering to a subject in need of treatment an amount of liposomes as disclosed herein.

Yet further, in accordance with a third aspect, there is provided a pharmaceutical composition comprising a physiologically acceptable carrier and as active ingredient liposomes as disclosed herein.

In addition, there is provided in accordance with a fourth aspect, the use of the liposomes as defined herein for the preparation of a pharmaceutical composition.

Finally, in accordance with a fifth aspect, there is provided by the present disclosure a kit comprising a composition containing liposomes as disclosed herein, and instructions for use of the composition of liposomes for executing a therapeutic treatment on a subject in need thereof.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1E are in vitro results including a graph (FIG. 1A) showing binding of hyaluronic acid targeted multilamellar liposomes (HA-L) to monolayers of RAW264.7 cells; a fluorescence image (FIG. 1B) of rhodamine/HA-L bounded to cultured macrophage surface (arrows), peritoneal macrophage obtained from Csf1R-icre-Rosa(26)TdTomato-EGFP Rosa mouse; TNF-α secretion (FIG. 1C); IL-12 secretion (FIG. 1D); and HO-1 expression in LPS-activated macrophages (FIG. 1E);

FIGS. 2A-2E are images showing active in vivo targeting of HA-L in mice with Myocardial Infarction (MI), FIG. 2A, 2C to 2E—infarcted heart, 2C to 2E—higher magnification of regions within the infarct heart, FIG. 2B—normal heart of sham mice; localization of HA-L is shown by arrows marking the liposomes' zone;

FIGS. 3A-3F are images of in vivo results of mice subjected to MI and were treated with either HA-L or with saline (control mice), the results show no targeting of HA-L to lungs (FIG. 3A), liver (FIG. 3C), and spleen (FIG. 3E), similar results were observed when the mice were administered only with saline as shown in FIGS. 3B (lung), 3D (liver) and 3F (spleen);

FIG. 4A-4H are images showing in vivo targeting of HA-L in apoE KO atherosclerotic mice, FIG. 4A shows active targeting of HA-L (arrows) to atherosclerotic plaques in aorta of apoE KO mice, FIGS. 4C to 4H show no in vivo targeting of HA-L in liver (FIG. 4C), spleen (FIG. 4E), and lung (FIG. 4G) of apoE KO mice, whereas FIGS. 4B, 4D, 4F and 4H are from apoE KO mice injected with saline. Localization of HA-L is shown by arrows marking the liposomes' zone at the aortic plaques (white arrows);

FIG. 5A-5C are images showing in vivo HA-targeted liposomes encapsulating an MRI contrast agent iron oxide nanoparticles (IONP/HA-L) to infarcted heart of pig. FIG. 5A is an MRI image of the pig's heart, FIGS. 5B and 5C are histological images of the infracted heart of FIG. 5A with the liposomes presence in the heart being identified by arrows marking the liposomes' zone;

FIGS. 6A-6D are histological images of kidney (FIG. 6A), liver (FIG. 6B), lungs (FIG. 6C) and spleen (FIG. 6D) from infarcted pig's after receiving HA-targeted liposomes encapsulating as an MRI contrast agent iron oxide nanoparticles (IONP/HA-L), the figures show no iron staining in these organs;

FIG. 7 is a bar graph showing cell viability of of LPS-activated RAW264.7 cells treated with: hemin-free HA-MLV, hemin-free regular multilamellar vesicles (RL-MLV), free hemin, hemin/RL-MLV and hemin/HA-MLV with hemin dose of 50 μg/ml and liposome dose of 4 mM lipid, each bar is an average of 6 wells and the error bars are the standard deviations;

FIGS. 8A-8F are graphs showing changes in proportion between reparative (M2) macrophages and pro-inflammatory (M1) macrophages in samples obtained from mice peritoneum (FIGS. 8A-8D) or cardiac macrophages (FIGS. 8E-8F), demonstrating that hemin/HA-MLV (2 mg hemin/kg body and 50 mg lipid/kg body) increased M2/M1 proportion, and decreased pro (M1)- and anti (M2)-inflammatory peritoneal macrophages;

FIGS. 9A-9R are bar graphs showing that hemin-HA-L (2 mg hemin/kg body and 50 mg lipid/kg body) attenuated left ventricle (LV) reimmunomodeling and improved heart function as indicated in the following parameters LV diastolic area (FIGS. 9A and 9B) and systolic area (FIGS. 9C and 9D), as well as diameters (FIGS. 9E to 9H), fractional shortening (FIGS. 9I and 9J), ejection fraction (FIGS. 9K and 9L), fractional area change (FIGS. 9M and 9N), AW thickness, diastole (FIGS. 9O and 9P), AW thickness, systole (FIGS. 9Q and 9R).

FIGS. 10A-10E are two dimensional echocardiography images seven days after MI showing effect on LV conicity index after treatment with Hemin/HA-L (FIG. 10A), empty/regular liposomes (FIG. 10B), free hemin (FIG. 10C), saline (FIG. 10D) and a corresponding graph of the % of conicity index change (FIG. 10E) the conicity index being calculated as the ratio between apical diameter to mid diameter of the LV in long axis (as shown). The p value was based on Kruskal-Wallis test.

FIGS. 11A-11B show the improvement in heart function measured by speckle tracking based stain as evident from the representative radial strain curves after MI of each indicated treatment from the change (%) in the radial anterior apex strain measured 7 days after MI (FIG. 11B) or from the change (%) in the longitudinal anterior mid strain 30 days after MI (FIG. 11C) following the different treatments.

FIGS. 12A-12B are graphs showing the change (%) in the anterior wall thickness 30 days after MI in systole (FIG. 12A) and diastole (FIG. 12B) following the different treatments.

FIGS. 13A-13E are representative hematoxylin-eosin staining of each treatment, hemin/HA-L (FIG. 13A), empty liposomes (FIG. 13B), free hemin (FIG. 13C) and saline (FIG. 13D) and a graph presenting the percentage (%) of the scar out of the LV area following the corresponding treatments (FIG. 13E).

FIG. 14 is a bar graph showing the impact of drug free regular multilamellar liposomes (no HA, Drug-free RL-L) and drug free hyaluronan multilamellar liposomes (Drug-free HA-L) on intracellular concentration of active HO-1 in NAR cells with Control being the basal HO-1 level in un-treated cells. Three liposome doses were used 4 (light gray bars), 10 (white bars) and 21 (gray bars) mM lipid, respectively. Each bar is an average of 6 wells, and the error bars are the standard deviations. Statistical significance was evaluated by student t test (two tail, unequal variance) for RL vs. control and for HA-L vs. control. *p<0.05, **p<0.003, ***p<0.0001.

FIG. 15 is a bar graph showing the impact of free and liposomal hemin on intracellular concentration of active HO-1 and on viability, in NAR cells (control and drug-free HA-L are as defined in FIG. 17). The “O” denotes cell viability (average of 6 wells) with the error bars representing standard deviations: the light-gray bars are the HO-1 concentration for the given wells (average of 6 wells), the error bars representing the standard deviations. The data of the gray bars are normalized to 100% cell viability. Statistical significance was evaluated by student t test (two tail, unequal variance), for HO-1 activity free hemin, drug-free HA-L and hemin/HA-L were compared, each, vs., control. For viability, hemin/HA-L was compared vs. control ***p<0.0003.

FIG. 16 include two images of tumor area from two different mice.

FIGS. 17A-17B are graphs showing the change in the animal weight (FIG. 17A) and tumor volume (FIG. 17B), with time and treatments. Each point is an average of all animals in the group, and the error bars are the SEM. The arrows in FIG. 17B indicate the days of injection; filled squares represent animals receiving drug-free HA-L (n=7); circles represent animals receiving free hemin (n=8) and open squares represent animals receiving hemin/HA-L. Doses were: 2 mg/Kg body hemin and 125 mg/Kg body lipid (liposomes). Statistical significance was evaluated by student t (unequal variance, two tail) for hemin/HA-L vs. drug-free hemin. *p<0.03, **p<0.014.

### DETAILED DESCRIPTION OF EMBODIMENTS

The present invention is based, inter alia, on the finding that while free hemin, or drug free hyaluronan (hyaluronic acid, HA) had a small and insignificant effect on TNF-α secretion, hemin-encapsulating hyaluronan-liposomes (HA-L) had a highly significant effect, almost abolishing the TNF-α secretion.

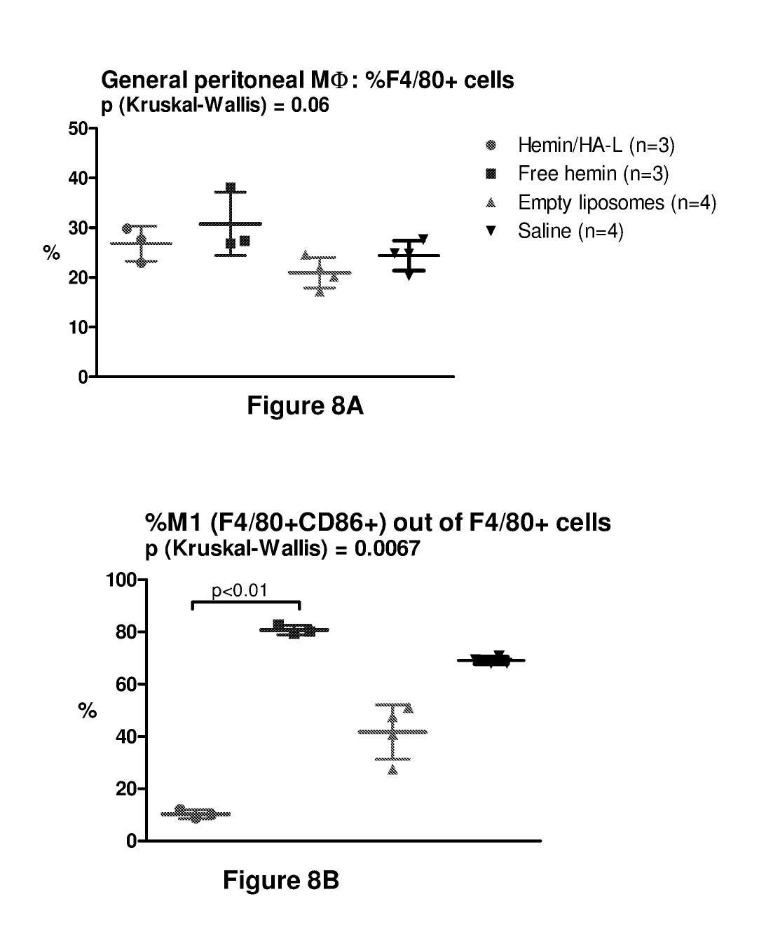

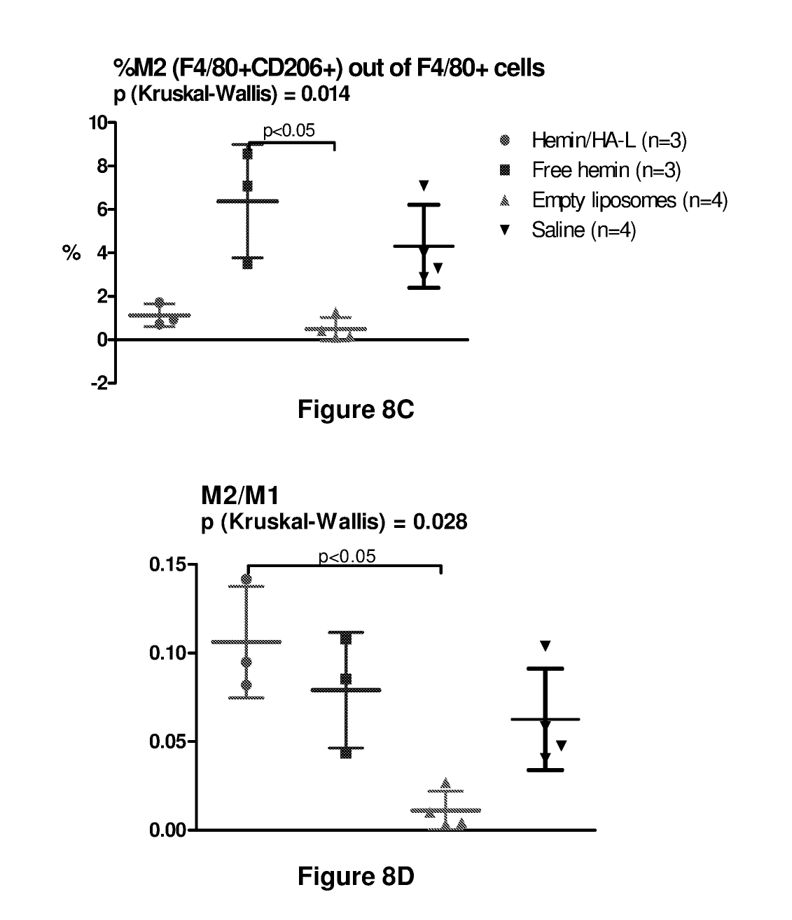

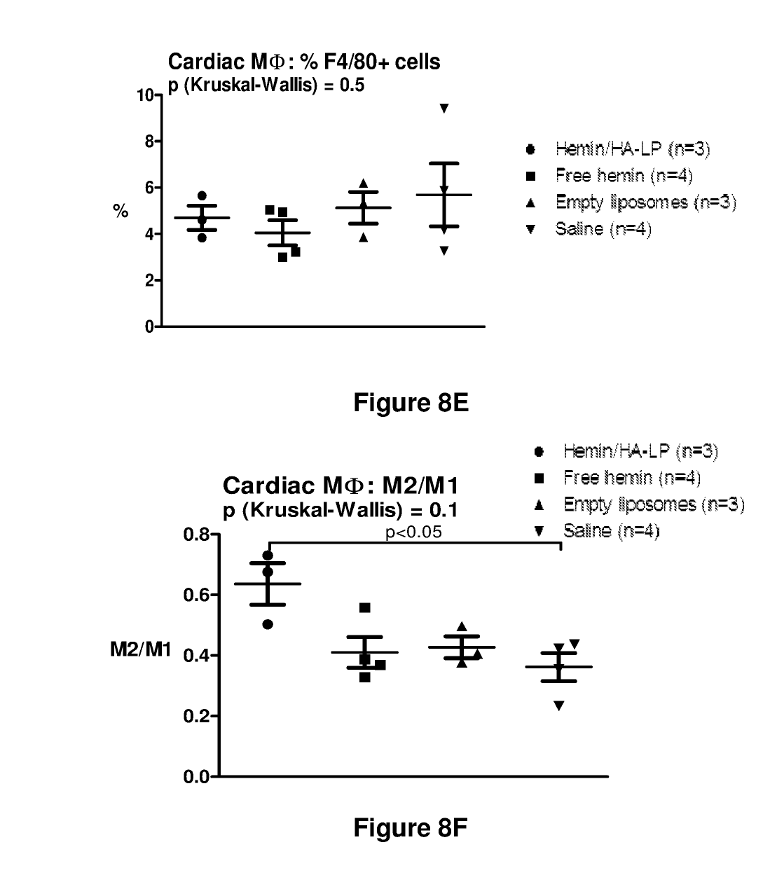

Further, as evident from the enclosed non-limiting examples, the hemin-encapsulating HA-L exhibited at low lipid concentration, high efficacy as an immunomodulatory treatment (reducing inflammation, i.e. as an anti-inflammatory agent), with particular suitability for treatment of non-infectious inflammatory conditions. The effect, shown for example, in FIG. 8, was much greater than the sum of effects of the free drug and empty hyaluronan-liposomes (HA-L) that may suggest, without being bound thereto, some synergistic effect between the iron containing complex (hemin) and the targeted liposome. This finding let to a first aspect of the present disclosure referred to herein as the immunomodulatory aspect as further discussed hereinbelow.

Further, as evident from the enclosed non-limiting examples, the high dose hemin-encapsulating HA-L exhibited at high lipid concentration an anti-cancer effect without affecting viability of non cancerous cells. This finding let to a second aspect of the present disclosure referred to herein as the anti-cancer aspect as further discussed herein below.

One unique feature of the anti-cancer aspect is that the liposomes “hit” two tumor targets with the same treatment (hemin/HA-L at the high liposome doses). The two targets are the cancer cells per se and the tumor-associated macrophages in the tumor microenvironment, as shown in the experimental section herein. Without being bound by theory it is believed that also the cancer-associated fibroblasts (CAFs) are targeted by the liposomes as these also have CD44 and HO-1.

The components of the liposomes employed for the immunomodulatory aspect and the anti cancer aspect are essentially the same with the difference residing mainly in the concentration of the liposomes' lipids concentration, being at a lower level when an immunomodulatory effect is desired, as further discussed below.

Based on the inventors' findings, there is now provided, a liposome (liposomes) being defined by a liposome membrane and an intraliposomal compartment/core having encapsulated in the intraliposomal compartment an iron complex comprising an iron moiety and a macromolecule; and the liposome having a targeting moiety anchored externally to the membrane.

The term “liposome” is used herein to define a bilayered vesicle, with the meaning acceptable in the art. The liposomes's membrane is a lipid bilayer membrane formed from a variety of liposome forming lipids. The lipid bilayer defines an inner liposomal core typically enclosing drugs or other active ingredients. This inner core is referred to in the art as the intraliposomal compartment. The lipid bilayer also defines an outer surface where the outer membrane of the bilayer faces the surrounding environment. This outer membrane may carry a variety of moieties, such as targeting moieties, for directing the liposomes to a desired target site. In this context, when referring to the targeting moiety being anchored at the outer surface of the liposome's membrane it is to be understood that the targeting moiety has a lipid tail being at least embedded to the external membrane of the lipid bilayer. In some embodiments, the targeting moiety is bound (covalently or non-covalently) to a lipid forming part of the lipid bilayer of the liposome. In some embodiments, the targeting moiety is bound (preferably covalently) to phosphatidyl ethanolamine (PE), the latter forming part of the bilayer.

The liposome-forming lipids may be defined by their capability to form into a bilayer. In some embodiments, the “liposome forming lipids” are primarily glycerophospholipids and sphingomyelins. The glycerophospholipids have a glycerol backbone wherein at least one, preferably two, of the hydroxyl groups at the head group is substituted by one or two of an acyl, alkyl or alkenyl chain, a phosphate group, or combination of any of the above, and/or derivatives of same and may contain a chemically reactive group (such as an amine, acid, ester, aldehyde or alcohol) at the head group, thereby providing the lipid with a polar head group. The sphingomyelins consists of a ceramide unit with a phosphorylcholine moiety attached to position 1 and thus in fact is an N-acyl sphingosine. The phosphocholine moiety in sphingomyelin contributes the polar head group of the sphingomyelin.

In some embodiments, the acyl chain(s) of the liposome forming lipids are typically between 14 to about 24 carbon atoms in length, and have varying degrees of saturation being fully, partially or non-hydrogenated lipids. Further, the lipid may be of natural source, semi-synthetic or fully synthetic lipid, and neutral, negatively or positively charged.

Examples of liposome forming glycerophospholipids include, without being limited thereto, glycerophospholipid, phosphatidylglycerols (PG) including dimyristoyl phosphatidylglycerol (DMPG); phosphatidylcholine (PC), including egg yolk PC, soy PC, dimyristoyl phosphatidylcholine (DMPC), 1-palmitoyl-2-oleoylphosphatidyl choline (POPC), hydrogenated soy phosphatidylcholine (HSPC), distearoylphosphatidylcholine (DSPC); phosphatidic acid (PA), phosphatidylinositol (PI), phosphatidylserine (PS), phosphatiydyl ethanolamine (PE).

In some embodiments, the liposome forming lipid is phosphatidylcholine, more specifically, soy PC (SPC).

At times, the liposome forming lipids may also include cationic lipids (monocationic or polycationic lipids). Cationic lipids typically consist of a lipophilic moiety, such as a sterol or the same glycerol backbone to which two acyl or two alkyl, or one acyl and one alkyl chain contribute the hydrophobic region of the amphipathic molecule, to form a lipid having an overall net positive charge. Preferably, the headgroup of the lipid carries the positive charge. The cationic lipids may be used for conjugating therewith the targeting moiety, although the targeting moiety may also be conjugated to neutral and other lipids.

The vesicle-forming lipids may be combined with other lipids. This combination with other lipids may be selected to achieve a specified degree of rigidity, to control the stability of the liposome in serum and to control the rate of release of the entrapped agent in the liposome. At times, it is required that the liposome forming lipids provide rigidity to the resulting membrane, so as to prevent undesired leakage of the iron complexes from the liposomes.

Accordingly, in some embodiments, the liposome's membrane may include lipids which are not considered liposome forming, but contribute to the liposomes' characteristics, such as stability. In some embodiments, the liposome's membrane comprises at least one of a lipid selected from the group consisting of glycolipids and sterols. In some embodiments, the liposomes' membrane comprises cholesterol. In yet some embodiments, the amount of sterol, such as cholesterol, will not exceed 40% out of the total lipid content (weight) in the liposome. In some other embodiments, the sterol will be in the range of between 0% to 40% w/w; at times, in the range of 5% to 40%.

The lipid based vesicles (i.e. liposomes) can be prepared by various methods known in the art. One procedure involves dissolving a mixture of at least the liposome-forming lipids in a suitable organic solvent and evaporating the organic solvent in a vessel to form a thin film. The film is then converted to the liposomes by rehydrating with an aqueous medium (that may include the drug to be encapsulated in the liposomes). In an alternative method, the lipid film may be rehydrated without the drug and then downsized (e.g. by sonication) to achieve a size distribution of the liposomes within a desired range (preferably essentially uniformly sized).

In some embodiments, the liposome is a multilamellar vesicles (MLV), which may be prepared e.g. by solvent injection, lipid hydration, reverse evaporation, freeze drying or by repeated freezing and thawing.

In some other embodiments, the liposomes are unilamellar vesicles. In some embodiments, the unilamellar vesicles are; small (<100 nm), in some other embodiments, the unilamellar vesicles are large (>100 nm) unilamellar vesicles (SUV or LUV, respectively). The unilamellar vesicles can be prepared by sonication, extrusion through polycarbonate filters having a defined pore size, by a French pressure cell, i.e., by passing MLV through small orifice under high pressure, or by solvent injection methods, with solvents such as ethers or alcohols.

In some other embodiments, the liposomes are stable plurilamellar vesicles (SPLV).

In yet some other embodiments, the liposomes are oligolamellar vesicles (OLV) whether prepared by detergent removal using dialysis, column chromatography, bio-beads SM-2, by reverse phase evaporation (REV).

The liposomes can also be giant multivesicular vesicles (MVV or GMVV, U.S. Pat. No. 6,162,462) which are at least 1 microns in diameter, and may be prepared by vortexing a lipid film with an aqueous solution of a suitable salt (e.g. ammonium sulfate), homogenizing the resulting suspension to form a suspension of small unilamellar vesicles (SUV), and repeatedly freeze-thawing said suspension of SUV in liquid nitrogen followed by water to form the MLV. All these and other methods of liposome preparation, are known in the art.

In some embodiments, the liposomes are MLV. Multilamellar liposomes have a size distribution in the micron range; in some embodiments, between 0.5 to 4 microns, or 1 to 3 microns; or even 1-2 microns in diameter.

In some embodiments, the liposomes are unilamellar liposomes which have a size distribution between 60 nm to 10 μm. In some other embodiments, the size distribution is between 100 nm to 600 nm, between 150 nm to 400 nm or between 200 nm to 300 nm.

In the context of the present disclosure the liposomes encapsulate (enclose) in their intraliposomal compartment at least the iron complex. As used herein, the term “iron complex” denotes a chemical macromolecule (typically composed of repeating smaller units) that carries an iron atom or ion. In some embodiments, the macromolecule is a macrocycle (cyclic macromolecule) defined by IUPAC as a cyclic macromolecule or a macromolecular cyclic portion of a molecule. These macrocyclic compounds are typically polydentate and present a central area that can bind to a metal atom or ion in two or more coordinates of the macromolecule to thereby form the metal complex (or iron complex). The binding may be covalent or non-covalent and may be regarded as a metal chelate. In the context of the present disclosure, when referring to a macromolecule it is to be understood as referring to an iron complex and vice versa.

In some embodiments, the macromolecule is a chelating macromolecule.

In some embodiments, the cyclic macromolecule comprises a porphine backbone. The term “porphine backbone” encompasses any macromolecule that is either porphine per se or a substituted derivative thereof. The non-substituted porphyrin and substituted porphines are known as porphyrins, and as appreciated by those versed in the art, there are many naturally occurring porphyrins, the most known being heme, and the specific porphyrin heme B, referred to at times as protoporphyrin IX (having 4 methyl, two vinyl, and two propionic acid substituents).

In some embodiments, the macromolecule is selected from the group consisting of porphine, porphyrin and protoporphyrin IX.

As noted above, the macromolecule holds an iron moiety, i.e. is in the form of an iron chelate with the macromolecule. When referring to a chelate it is to be understood as the chemical structure involved in the formation or presence of two or more separate coordinate bond between the polydentric macromolecule a single central atom. In some embodiments, the macromolecule is porphrin bound to the iron moiety via the four nitrogen atoms of the tetrapyrrole of this macrocycle.

In some embodiments, the iron moiety is an iron ion. In yet some further embodiments, the iron ion is selected from the group consisting of Fe₂³⁺ and Fe²⁺.

The iron complexes in accordance with the present disclosure do not include iron oxides and/or iron oxide nanoparticles. Such nanoparticles are excluded from the scope of the present disclosure.

In some embodiments, the iron complex is hemin, namely, an iron-porphyrin chelate.

The liposomes also carry a targeting moiety at the outer surface of the liposomes. The targeting moiety may be a small molecular weight compound (e.g. mono or disaccharide) as well as intermediate or large macromolecule (e.g. polypeptide, protein, polysaccharide) associated to the membrane with at least the targeting portion thereof being exposed at the outer surface of the liposomes. The association of the targeting moiety may be either by covalent binding to a member of the membrane or non-covalent interactions with the outer membrane of the liposomes (e.g. adsorption).

In some embodiments, the association of the targeting moiety with the outer surface of the liposomes is by binding the targeting moiety to the outer membrane. When referring to “binding” it is to be understood any one of the following: (i) the targeting moiety includes or is modified to include a tail, typically lipophilic (or lipid) tail that is embedded in the outer lipid layer of the liposome's membrane (i.e. the lipid tail of the targeting moiety is in parallel with the lipids forming the outer layer of the lipid bilayer forming the liposome's membrane); (ii) the targeting moiety includes a functional group that is capable of binding (covalently or non-covalently) to a lipid forming part of the liposomal lipid bilayer; (iii) the targeting moiety is capable of being stably adsorbed onto the surface of the lipid bilayer, e.g. by non-covalent interactions, e.g. hydrophobic, van der waals, electrostatic etc.

In some embodiments, the targeting moiety is a saccharide-based moiety and in one particular embodiment, the targeting moiety is a high molecular weight hyaluronic acid (HA, (C₁₄H₂₁NO₁₁)ₙ, n being at 1,000, at times, at least 1,500, 2,000, 2,500; 3,500, 4,000, 5,000, in these ranges or n being even greater. HA coating/targeting may assist in specific delivery of the liposomes to the hyaluronan receptors (particularly the CD44 family) at leukocyte (particularly, but not exclusively macrophage), endothelial, smooth muscle, and mesenchymal cell surface. In fact, as shown in the non-limiting Examples below, liposomes conjugated with HA showed selective accumulation in injured tissues. Specifically, liposomes conjugated with HA were shown to selectively accumulate in hearts of mice with MI and in the aorta atherosclerotic plaque of apoE KO mice. Interestingly, accumulation of liposomes conjugated with HA was not observed in other tissues (such as lung, spleen and liver) of mice induced with MI and/or in apoE KO mice and also in hearts of control mice (not injured).

Hyaluronic acid can be associated to the liposome by various methods. In some embodiments, the hyaluronic acid is conjugated to a lipid (forming a lipid tail to HA) that then forms part of the lipid layer. Conjugation of HA to lipids are known in the art, and some non-limiting examples include the conjugation of HA with amine residues of lipids, such as phosphatidyl amine (PE) or any known derivative thereof (i.e. any fatty chain attached) as long as the amine is free to cross-link (conjugate) with the HA. Non-limiting examples include, PE per se, 1,2-dioleoyl-sn-glycero-3-phosphoethanolamine (DOPE) and Dipalmitoyl-sn-glycero-3-phosphoethanolamine (DPPE) and others.

In some embodiments, the liposome comprises a combination of liposome forming lipids and lipids that do not spontaneously form into liposome (non-liposome forming lipids), the latter including a sterol. In one embodiment, the liposome is composed of a combination of PC derivative, PE and cholesterol. In yet some further embodiments, the liposome comprises soy phosphatidylcholine (SPC), Cholesterol and 1,2-Dipalmitoyl-sn-glycero-3-phosphoethanolamine (DPPE).

In one embodiment, the liposome comprises soy phosphatidylcholine (SPC):Cholesterol:1,2-Dipalmitoyl-sn-glycero-3-phosphoethanolamine (DPPE) at the mole ratio of 55:40:5.

In some other embodiments, the liposomes comprise about 5% w/w, sterol (in particular cholesterol) in the range of 0-40% and liposome forming lipid (in particular PC or PC based) in the range of 55-95%.

In some embodiments, the liposomes' composition is such that the effect obtained by the combination of a targeting moiety exposed at the liposomes' surface, and an iron complex encapsulated within the liposomes is greater than the sum of the effect obtained by administering a free iron complex and empty (targeted) liposomes. The effect is greater at least by a factor of 1.2, 1.5 or 1.8, at times, twice, thrice or even greater.

It has been determined by the inventors that the ratio between the iron complex and the lipids must be such that the lipid concentration is at least 5 times, at times, at least 10, or even 25, 30, 40, 50 or even 55 times greater than that of the iron complex.

In accordance with the immunomodulatory aspect, the additional requirement is that the lipid concentration remains at a non-toxic level for the particular targeted cell or tissue.

In some embodiments of the immunomodulatory aspect, the molar ratio between the lipid to the iron complex such as hemin, in the range of 5-120.

In accordance with the anti-cancer aspect, the additional requirement is that the lipid concentration is such that it also affects viability of at least the target cancerous cell.

In some embodiments of the anti-cancer aspect, the molar ratio between lipid to the iron complex, such as hemin, in the range of ≧120. At times, no more than the ratio that would cause deleterious damage to normal cells. At times, the range is 120-1,400.

As noted herein, the HA coating/targeting assist in specific delivery of the liposomes to the hyaluronan receptors, and in particular to cells expressing CD44 family).

In accordance with the immunomodulatory aspect, the liposomes targeted to CD44 at the macrophage surface comprise iron complex and lipid concentration that while being sufficiently low (as defined) to cause no effect on viability of the macrophage, it is yet sufficient to induce anti-inflammatory action of the macrophages.

With respect to the immunomodulatory aspect, the inventors have unexpectedly found that surface targeted (e.g. HA-modified) liposomes encapsulating hemin have a greater effect on reducing TNF-α and/or IL-12 secretion as compared to the effect of free hemin or hemin encapsulated in regular liposomes (non-targeted/non-surface modified). Specifically, as shown in the non-limiting examples, free hemin did not affect TNF-α secretion and hemin encapsulated in regular liposomes showed only a small and non-significant reduction in TNF-α section and/or IL-12 secretion; while a significant and unexpected reduction in TNF-α section was observed with the hemin-encapsulated HA targeted liposomes. In addition, a significant increase in the ratio of M2/M1 (see further below) was observed with the hemin-encapsulated-HA-modified liposomes as compared to that obtained with free hemin or hemin encapsulated in regular liposomes (non-targeted).

Macrophages constitute a heterogeneous population and can exhibit pro-inflammatory cytokine profiles and cytotoxic activities, as well as anti-inflammatory and tissue repair activities. They play an important role in tissue homeostasis and remodeling and are also potent immune regulators.

Macrophage is the dominant cell type in various acute conditions, such as myocardial infarction, in which macrophages control the initiation, maintenance, and resolution of inflammation. For example, after myocardial infarct (MI), the heart tries to compensate itself by left ventricular remodeling. One of the earliest phases after MI involves acute inflammation leading to fibrosis and scar formation. Neutrophils and monocytes are the first to infiltrate the infarct. Monocytes become macrophages, which then take part in the acute inflammation as well as in the following healing and repair phases. Injection of activated macrophages into an infarct is associated with improved vascularization, myofibroblast accumulation, scar thickening and accumulation of resident macrophages which, in turn, contribute to infarct healing in rats.

Monocytes and macrophages display remarkable plasticity and can change their physiology in response to environmental cues. Some macrophages exhibit a pro-inflammatory cytokine profile and cytotoxic activity (“classical activation state”—M-1 polarization), whereas others show an anti-inflammatory profile and tissue repair activity (“alternative activation state” or “anti-inflammatory and reparative macrophage phenotype”—M-2 polarization). In a mouse model of myocardial infarction (MI), M-1 macrophages digested damaged tissue during the first days after MI, whereas M-2 macrophages subsequently promoted healing via myofibroblast accumulation, angiogenesis, and deposition of collagen.

A macrophage in a reparative or anti-inflammatory state refers to macrophages displaying a Th2-like phenotype. Sometimes such state is also referred to as M-2 state or M-2 subtype and is known as promoting ECM construction, cell proliferation, and angiogenesis, this being opposed to the classically activated macrophages (sometimes referred to as M-1 state) that exhibit a Th1-like phenotype, promoting inflammation, extracellular matrix (ECM) destruction, and apoptosis.

The classically activated macrophages are known to increase inflammation and tissue injury while the alternatively activated macrophages are known to suppress inflammation and promote wound healing.

As shown herein below, administration of liposomes encapsulating iron complexes with low concentration of lipid (e.g. less than 5 mM, as described hereinbelow) improved myocardial protection, infarct healing, repair and regeneration after myocardial infarction. Specifically, it has been shown to switch classically activated macrophages into alternative reparative macrophages that produce therapeutic cytokines.

It was thus concluded that targeted liposomes encapsulating iron complexes and having said low lipid concentration can be used as a therapeutic tool to treat non-infectious inflammatory disorders by switching pro-inflammatory, classically activated macrophages (M1) to anti-inflammatory, alternatively activated (M2), reparative macrophages.

These findings led the inventors to the understanding that the liposomal formulations having a low lipid concentration have an immunomodulation beneficial effect and can be used for treating inflammation and specifically non-infections inflammation and more specifically macrophage-mediated inflammation.

In the context of the present disclosure a “low lipid concentration” denotes a concentration that provides a lipid to macromolecule molar ratio that is less than 120. In some embodiments, the lipid concentration is equal or less than 5 mM. In some further embodiments, a low lipid concentration refers to a lipid:macromolecule molar ratio of between 5 to 120, which corresponds to a concentration between 0.5 mM and 5 mM, at times, between 1 mM and 4 mM, further at times, between 2 mM and 4 mM. In this context the lipid refers to both liposome forming lipids and non-liposome forming lipids (e.g. sterol).

The low lipid concentration was found to be effective in treating non-infectious inflammatory disorders. When referring to “non-infectious inflammatory disorders” being used interchangeably with the term “sterile inflammatory disorders” it is to be understood as encompassing any immune response that is not related to activation of the immune system, e.g. by an infection. Such non-infectious inflammatory disorders denote any disorder which the activation of macrophages or activated macrophages play a role such as auto-immune disorders and inflammatory disorders which are not infection related, i.e. non-pathogenic, caused by other than an infectious agent (e.g. auto-antigen, hypersensitivity, wound). Illustrating but not limiting examples of such activated macrophage-related, non-infectious inflammatory disorders are inflammatory diseases of the gastrointestinal tract such as Crohn's disease, inflammatory bowel disease, gastritis, colitis, ulcerative colitis, colon irritable, gastric ulcer and duodenal ulcer, inflammatory diseases of the skin such as psoriasis, inflammatory diseases of the respiratory system such as asthma, allergic rhinitis or chronic obstructive pulmonary disease (COPD), pulmonary fibrosis, sarcoidosis, inflammatory diseases of the musculoskeletal system such as rheumatoid arthritis, osteomyelitis, osteoporosis, or neuritis, systemic sclerosis, inflammatory diseases of the kidneys such as glomerulonephritis, renal ischemia, or renal inflammation; inflammatory diseases of the nervous system such as multiple sclerosis, Alzheimer's disease and HIV-1-associated dementia; autoimmune diseases such as diabetes, type 1 and 2 diabetes mellitus and graft versus host reaction; infectious disease such as nephritis, sepsis, septic shock, endotoxic shock, adult respiratory distress syndrome; inflammatory conditions of the cardiovascular system, such as myocardial infarction, myocarditis, atherosclerosis, hypertensive cardiomyopathy, atheroma, intimal hyperplasia or restenosis.

The non-infectious inflammatory disorders also denote tissue healing and repair. The tissue may be injured or damaged as a result of ischemia or necrosis.

Non-limiting examples of tissue damage may include infracted myocardium, myositis, myocarditis, myocardial fibrosis, glumerulonephritis, diabetic nephropathy, kidney infarct, glomerular sclerosis, stroke, liver injury, brain injury, pulmonary fibrosis, ischemic limb, athrosclerosis vascular disease, chronic ulcer.

The non-infectious inflammatory disorders may be an acute non-infectious inflammatory disorder or chronic a non-infectious inflammatory disorder.

It is specifically noted that the non-infectious inflammatory disorders exclude any pathogenic, e.g. bacterial, mediated inflammation (e.g. due to pathogenic infection).

In some embodiments, the non-infectious inflammatory disorders may be involved in a condition selected with tissue injury, infarct, trauma, and necrosis. According to some embodiments, the non-infectious inflammatory disorders are associated with a damaged tissue condition, for example healing a wounded tissue.

In some additional or alternative embodiments, the non-infectious inflammatory disorders are of the cardiovascular system, such as, and without being limited thereto myocardial infarction (MI), acute MI (commonly known as heart attack), myocarditis and atherosclerosis.

In one embodiment, the non-infectious inflammatory disorder is myocardial infarction. As may be appreciated after myocardial infarction (MI), uncontrolled activation of pro-inflammatory macrophages accelerated left ventricular (LV) remodeling and dysfunction are often observed. As shown herein below, an improvement in different parameters after MI was observed in mice treated with hemin/HA-L compared to control.

In some embodiments the treatment with the liposomes according to the immunomodulatory aspect involves promoting macrophages to shift to a reparative or anti-inflammatory state thereby treating the non-infectious inflammatory disorders. In this context, the non-infectious inflammatory disorders may be a type inflammation medicated by activated macrophage.

As noted above, the inventors have found that there is significance to the ratio between the lipid content and the iron complex content on the effect on TNF-α and/or IL-12 secretion and/or HO-1 intracellular levels as shown herein in the following examples being part of the present disclosure. To this end and in accordance with the immunomodulatory aspect of the present disclosure, the liposome when in a liposomal formulation, comprises lipid to macromolecule molar ratio that is less than 120, or a lipid concentration between 1 and 5 mM and macromolecule (iron complex) concentration of 10-50 μg/ml (approximately 0.015 mM and 0.078 mM, respectively). These concentrations are within the lipid to macromolecule molar ratios defined herein with respect to the immunomodulatory aspect, i.e. within the range of 5-120.

In accordance with the anti-cancer aspect, the liposomes targeted to CD44 at the surface of cancer cells, have a lipid concentration sufficiently high to cause deleterious effect on the viability of at least the targeted cancerous cells. In accordance with this aspect, a high lipid concentration denotes one that provides a lipid to macromolecule molar ratio greater than 120, albeit, preferably without deleterious damage to normal cells. In some embodiments, the lipid concentration denotes any concentration above 5 mM. In some embodiments, the lipid concentration is above 10, 11, 12, 13, 14, 15, 16, 17, 18, 19, 20, 21, 22, 23, 24, 25 or even as much as 30 mM. At times, the lipid concentration is at most 22 mM. Within the range of these lipid concentrations, it is noted that the macromolecule concentration is as in the immunomodulatory aspect, i.e. macromolecule concentration of 10-50 μg/ml (approximately 0.015 mM and 0.078 mM, respectively).

In accordance with the anti-cancer aspect the liposomes are effective to result in a cytotoxic effect on the targeted CD44 expressing cells.

In some embodiments, the liposomes are used in a method of treating cancer, particularly cancer comprising cells expressing CD44.

The targeted CD44 expressing cells in the context herein are the CD44 expressing tumor cells and at times also tumor associated macrophages that support and assist the tumor in its proliferation. As appreciated by those versed in the art, the depletion of tumor associated macrophages from the tumor microenvironment is one of the approaches for tumor treatment and thus would be beneficial. Thus, in the context of the anti-cancer aspect, the high lipid doses would beneficially affect both the cancer cells themselves and the tumor-associated macrophages.

In some embodiments of the anti-cancer aspect the liposomes are used for treating prostate cancer.

In some embodiments of the anti-cancer aspect the liposomes are used for treating breast cancer.

In some embodiments of the anti-cancer aspect the liposomes are used for treating colon cancer.

In some embodiments of the anti-cancer aspect the liposomes are used for treating pancreas cancer.

In some embodiments of the anti-cancer aspect the liposomes are used for treating brain cancer.

In some embodiments of the anti-cancer aspect the liposomes are used for treating skin cancer.

In some embodiments of the anti-cancer aspect the liposomes are used for treating melanoma.

In some embodiments of the anti-cancer aspect the liposomes are used for treating bone cancer.

In some embodiments of the anti-cancer aspect the liposomes are used for treating sarcoma.

In some embodiments of the anti-cancer aspect the liposomes are used for treating ovary cancer.

For therapeutic utilities, such as noted above, the liposomes may be formulated with a physiologically and/or pharmaceutically acceptable carrier. In some embodiments, the composition is used in a method for treatment the desired inflammatory disease, in some other embodiments, the composition is used in a method for treatment the desired cancer, specifically CD44 expressing cancer cells.

In the context of the present disclosure “treatment” is understood to refer to a desired pharmacological and physiological effect, including prophylactic (e.g. preventing or partially preventing the development of a disease as detailed above or the effect can be therapeutic in terms of healing, ameliorating or reducing severity of the disease. In some embodiments, treatment includes curing (partially or fully) of damaged (injured) tissue, tissue regeneration, tissue remodeling, reduction of tumor size or elimination of tumor, all being in accordance with the respective aspect of the present disclosure.

The amount of the iron complex within the targeted liposome and the lipid concentration (or lipid to macromolecule molar ratio) can be determined in appropriately designed clinical trials (dose range studies) and the person versed in the art will know how to properly conduct such trials in order to determine the amounts. As generally known, an effective amount depends on a variety of factors including the distribution profile of the liposomes within the body, a variety of pharmacological parameters such as half life in the body, undesired side effects, if any, on factors such as age and gender of the treated individual etc.

The targeted liposomes encapsulating the iron complex can be administered orally, subcutaneously (s.c.) or parenterally including intravenous (i.v.), intraarterial (i.a.), intramuscular (i.m), intraperitoneally (i.p), intranasal (i.n), and retroretinal, administration as well as by infusion techniques.

In some embodiments, the targeted liposomes encapsulating the iron complex are formulated with a physiologically acceptable carrier suitable for systemic delivery, i.e. by injection into the bloodstream.

In some other embodiments, the targeted liposomes encapsulated with iron complex are delivered locally by injection to the target site or its surrounding.

In some embodiments, the targeted liposomes encapsulating the iron complex are delivered orally.

In some embodiments, the targeted liposomes encapsulating the iron complex are delivered intra-tumoral, e.g. when the tumor is accessible.

In the context of the immunomodulatory aspect, the term “target site” is used to denote tissue or organ that is either inflamed (sterile inflammation); or tissue that is injured for example a wounded tissue or an ischemic tissue. In some embodiments of this aspect, the target site is a heart or portion thereof. In this context it is to be understood that the liposomes are targeted to macrophages (expressing CD44) that accumulate at the inflamed tissue.

In the context of the anti-cancer aspect, the term “target site” is used to denote cancerous tissue (including the tumor microenvironment) or cells expressing CD44.

In line with the above, the present disclosure also provides a method of treatment comprising administering to a subject in need of treatment an amount of liposomes as defined herein. In some embodiments, the treatment is of non-infectious inflammatory disorders as disclosed above. In yet some other embodiments, the treatment is of cancer, the cancer being of a type expressing CD44 (in the cancer cells and in the tumor microenvironment).

The liposomes of the present invention can be formulated or administered in combination with one or more other active agents, such as one or more anti-inflammatory or anti-cancer agents.

Examples of anti-inflammatory agents which can be combined with the liposomes include, without being limited thereto, steroidal anti-inflammatory drugs and non-steroidal anti-inflammatory drugs (NSAID), immune suppressive drugs. The additional agent can be administered to the subject before, concomitant or after administration of the liposomes as described herein. When administered together with the, the two (or more) may be in the same formulation or formulated in two different formulations.

Examples of anti-cancer agents which can be combined with any available drug known to those versed in the art (e.g. pharmacists, oncologist), these include, without being limited thereto conventional, low molecular weight chemotherapeutics, antibodies and nucleic acid based drugs (e.g. siRNA, miRNA).

The liposomes as described herein can form part of a therapeutic kit for use by a practitioner, e.g. a medical doctor, a nurse, or by a subject in need of the treatment. The kit can comprise the liposomes as described herein within a physiologically acceptable carrier, or the liposomes and the physiologically acceptable carrier to be combined (mixed) prior to use, and instructions for use of the liposomes with the physiologically acceptable carrier for administering to the subject, typically by injection. The kit can comprise a single dosage unit of the liposomes as described herein for single administration or may comprise multiple dosages of the particles, e.g. for multiple, sequential administrations according to a predefined schedule of treatment.

Finally, the present disclosure provides the use of liposomes as defined herein for the preparation of a pharmaceutical composition, also as defined herein.

The invention will now be exemplified in the following description of experiments that were carried out in accordance with the invention. It is to be understood that these examples are intended to be in the nature of illustration rather than of limitation. Obviously, various modifications and variations of these examples are possible in light of the above teaching. It is therefore, to be understood that within the scope of the appended claims, the invention may be practiced otherwise, in a myriad of possible ways, than as specifically described hereinbelow.

As used herein, the forms “a”, “an” and “the” include singular as well as plural references unless the context clearly dictates otherwise. For example, the term “a liposome” includes one or more liposomes. Further, as used herein, the term “comprising” is intended to mean that the composition include the recited liposomes, but not excluding other elements, such as physiologically acceptable carriers and excipients as well as other active agents. The term “consisting essentially of” is used to define the recited elements but exclude other elements that may have an essential significance on the desired treatment. “Consisting of” shall thus mean excluding more than trace elements of other elements. Embodiments defined by each of these transition terms are within the scope of this invention.

### SOME NON-LIMITING EXAMPLES

### Methods

### Preparation of HA-Targeted Hemin Encapsulated Liposomes and Use Thereof in Treating Myocardial Infarction

### I. Preparation of Drug-Free Hyaluronic Acid-Multilamellar Vesicles (HA-MLV)

Ia. RL-MLV (Regular MLV):

The liposomes were composed of soy phosphatidylcholine (SPC): Cholesterol:1,2-Dipalmitoyl-sn-glycero-3-phosphoethanolamine (DPPE) at the mole ratio of 55:40:5 and the total lipid concentration of 20 mg lipid/ml. The lipids were dissolved in ethanol and subjected to evaporation in a rotary evaporator under reduced pressure. The dry lipid film was incubated (in a shaker bath) with the swelling solution (0.1 M borate buffer at pH 9) for 2 hours at 65° C.

Ib. HA-MLV (HA-Targeted MLV):

Hyaluronan (HA) was dissolved in acetate buffer (0.1 M, pH 4.5) at the concentration of 2 mg/ml and pre-activated by incubation with the crosslinker EDC for 2 hours at 37°. The activated HA was mixed with the RL-MLV suspension at the ratio of 1:1 (v/v). This reaction mixture was incubated for 24 hours in a shaker bath at 37° C. [17-20]. The HA-MLV were freed from excess materials and by-products by centrifugation for 30 minutes at 4° C. and a g force of 160,850, followed by several successive washes and re-centrifugations in 0.1 M NH₄HCO₂, suspending the final pellets in this salt solution. The HA-MLV suspension was divided into aliquots of 100 μl or 500 μl and frozen for 2 hours at −80° C., followed by lyophilization. The resultant liposome powders were stored at −18° C. until further use. This produces powders of salt-free buffer-free liposomes, since the NH₄HCO₂ salt turns in the course of lyophilization into ammonium and CO₂ gases that are pumped out of the system.

### II. Preparation of Hemin-Encapsulating HA-MLV.

The lyophilized powder of the drug-free HA-MLV was rehydrated in an aqueous solution of hemin in PBS pH 7.6, and the system was incubated in a shaker bath for 24 hours, at 37° C.

### III. Preparation of Drug-Free RL-MLV and of Hemin-Encapsulating RL-MLV.

The processes were the same as described in parts I and II above for HA-MLV, with the following exception: In part Ib, the RL-MLV were incubated 1:1 with acetate buffer devoid of activated crosslinker.

It is noted that in the present disclosure “HA-L” is equivalently referred to by the abbreviation “HA-MLV”, of “HA-LP”, similarly, “Hemin/HA-L” is equivalently referred to by the abbreviation “Hemin/HA-MLV”, or “Hemin/HA-LP” and “RL-L” is equivalently referred to by the abbreviation “RL-MLV” or “RL-LP”.

### In Vitro Binding of Drug Free HA-Targeted Liposome (HA-L)

Two days prior to the experiment RAW264.7 cells were seeded onto 24-well plates and the experiment was initiated upon 95% confluency. The cell-growth media was replaced with 500 μl of serum-free medium containing the desired liposomes at increasing concentrations. Incubations were for 60 minutes at 37° C. Upon termination, the medium from each well was aspirated, and each well was washed three times with PBS. The cells from the control wells were lifted by trypsin and used to count the number of viable cells/well using the trypan blue method. The cells in all other wells were lysed with 1 N NaOH over night at 37° C., the content of each well collected and assayed for cell-associated liposomes. Liposome assay was by inclusion of trace [³H]Cholesterol in the formulation. Wells receiving medium alone served as controls.

### In Vitro Viability of Hemin Encapsulated HA-Targeted Liposome (Hemin/HA-L)

I. RAW264.7 cells were seeded onto 96 well plates, and the experiments were initiated 24 hours later, at semi-confluency. The regular (serum supplemented) cell growth media was replaced by the same media but containing one of the following treatments: free hemin, drug-free RL-MLV, HA-MLV, hemin-encapsulating RL-MLV and hemin-encapsulating HA-MLV. Wells receiving the regular cell growth media without any addition served as controls. Incubation time was 24 hours, at the end of which the experiment was terminated and cell viability was determined by the XTT or SRB methods. Both methods yielded similar results.

II. In addition, to confirm the ability of HA-MLV to target macrophages, peritoneal “green” macrophages from Csf1R-icre-Rosa(26)TdTomato-EGFP Rosa mouse were used (obtained from The Jackson Laboratory, USA). These cells allow direct fluorescent visualization of EGFP in monocytes and macrophages in live. ROSAmT/mG is a cell membrane-targeted, two-color fluorescent Cre reporter allele; expressing cell membrane-localized red fluorescence in widespread cells/tissues prior to Cre recombinase exposure, and cell membrane-localized green fluorescence in Cre recombinase expressing macrophages (and future cell lineages derived from these cells). The “green” (EGFP) macrophages were cultured with rhodamine encapsulated liposomes in order to visualize the binding of HA-MLV to the macrophage surface.

III. Further, the effect of hemin/HA-LP on TNF-α secretion levels from LPS stimulated RAW264.7 macrophages, on IL-12 cytokine secretion from peritoneal macrophages stimulated with LPS for 24 hours, and expression of HO-1 expression were measured.

### Mouse Model of Myocardial Infarction (MI)

Twelve week-old female Balb/C mice (20 gr.) were anesthetized with 2% isoflurane, and were surgically opened by left thoractomy through the fourth intercostal space to expose the beating heart. To induce MI, the left main coronary artery was permanently occluded with an intramural suture.

### Targeting Infarct Macrophages by HA-Liposomal Rhodamine (Rhodamine/HA-L)

To test liposomes efficacy, mice were allocated to either hyaluronan liposomes (HA-L) containing PE-rhodamine or saline, three days after MI. Injections were through mice tail-vein, containing 10 mg rhodamine in 100 μl HA-L volume, or 100 μl saline as control. Six hours after injection, mice were euthanized and their organs (heart, liver, lung and spleen) were taken for histological analysis.

### Peritoneal Macrophage Profile Following Hemin/HA-L Injection

To determine liposomal hemin effect on peritoneal and cardiac macrophages, hemin was formulated in HA-L. Hemin encapsulation efficiency was ˜100% at therapeutic dose levels. Mice were allocated with hemin/HA-L (2 mg hemin/kg body and 50 mg lipid/kg body), empty liposomes (HA-targeted, 50 mg lipid/kg body), free hemin (2 mg/kg body) or saline (100 μl) by intra-peritoneal injection one day after MI. Three days later, mice were euthanized and peritoneal macrophages were aspirated from abdominal cavity. Cells were counted and stained with an anti-mouse F4/80 (as general macrophage marker) and with either PE-conjugated anti-mouse CD86 (as M1 marker) or FITC-conjugated anti-mouse mannose receptor (CD206; as M2 marker). Cells were analyzed by FACS Calibur cytometer using CellQuest™ software.

### Echocardiography Examination

Following peritoneal macrophage profile characterization, a functional one week follow up study was set by echocardiography of mice after myocardial infarction.

Mice were anesthetized with 2% isoflurane, and echocardiograms were performed with a commercially available mouse echocardiography system (Vevo 2100, VisualSonics, Toronto, Canada) equipped with 30 MHz phased array. Transthoracic echocardiography was performed on all animals one day, one week and 30 days after MI. Hearts were imaged by 2-dimensional mode in the parasternal long- and short-axis views, through which the M-mode cursor was positioned perpendicular to the LV septum and posterior wall. An experienced technician, blinded to the treatment groups, performed all measurements that were averaged for 3 consecutive cardiac cycles.

### Statistical Analysis

Statistical analysis was performed with the GraphPad Prism version 5.00 for Windows (GraphPadSoftware, San Diego, Calif.). All variables are expressed as mean±SD.

### Results

### In Vitro Binding of HA-L

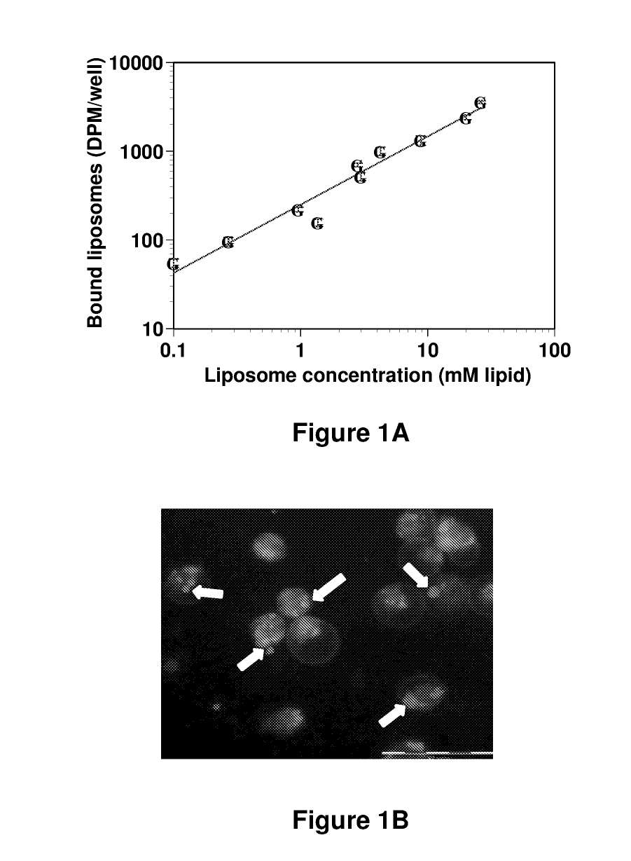

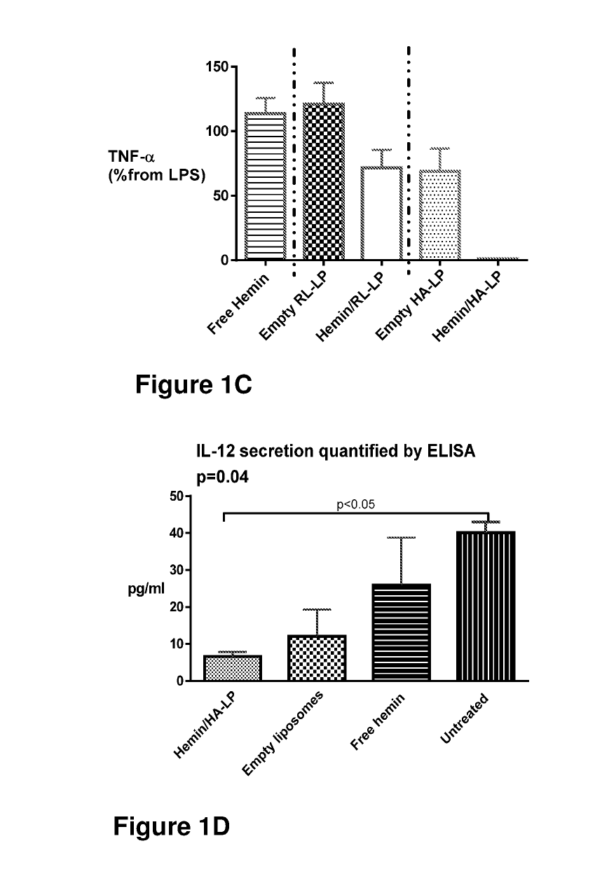

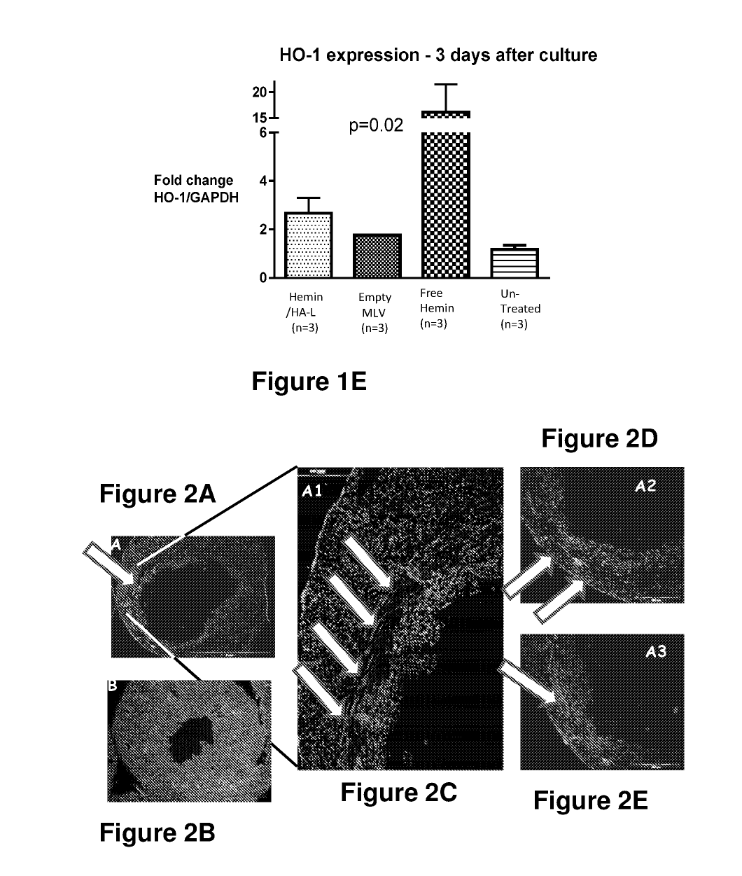

As shown in FIG. 1A, all liposome doses applied were within the range in which the quantity of cell-bound liposomes increases with the increase in liposome dose, namely below binding saturation.

FIG. 1B shows that markedly, HA-L is bound to the surface of peritoneal “green” macrophage, with and without internalization, one day after incubation.

In addition, the effect of hemin/HA-L on macrophage inflammatory and anti-inflammatory properties was evaluated. First, TNF-α secretion levels from LPS stimulated RAW264.7 macrophages was measured (FIG. 1C, “LP” denoting liposome). While free hemin and empty (free-drug) regular liposomes (RL-LP) showed high levels of TNF-α, hemin/RL-LP and empty HA-LP decreased its secretion. Interestingly, the most dramatic effect was of hemin/HA-LP, which decreased almost entirely TNF-α secretion (FIG. 1C). Next, mouse peritoneal macrophages were stimulated with LPS for 24 hours. After LPS removal, the cells were treated with either hemin/HA-LP, empty HA-LP, free hemin or saline. After three days in culture, hemin/HA-LP significantly decreased pro-inflammatory IL-12 cytokine secretion (FIG. 1D; p=0.04) and increased HO-1 expression within macrophages (FIG. 1E). Free hemin treated macrophages showed the highest HO-1 expression (FIG. 1E). Together, these findings demonstrated that a combination of hemin encapsulated in HA-LP has anti-inflammatory properties.

### In Vivo Targeting of Drug Free HA-L

### Fluorescently-Tagged HA-L in Infarcted Mice.

The fluorescent tag, PE-Rhodamine B, was included in the lipid composition of the HA-L. The criterion for successful active targeting had two parts: (1) high accumulation of HA-L liposomes at the target tissue, and (2) little or none targeting to other tissues, including, organs where regular liposomes tend to accumulate such as liver, spleen, kidneys and lungs.

To that end, HA-L were injected IV (mouse tail vein) at day 3 after induction of MI. Six hours after injection, mice were euthanized and hearts as well as other organs were cryo-sectioned to detect localization of the liposomes. As seen in FIGS. 2A-2E, the first part of the targeting criterion was achieved as accumulation of liposomes was observed in hearts undergoing MI (FIGS. 2A, 2C-2E). Specifically, FIG. 2A shows accumulation of fluorescently tagged HA-L in the heart of infracted mice, the zone of accumulation being marked by the arrow; FIGS. 2C to 2E show the enlargement of the marked areas in FIG. 2A where the liposomes have accumulated.

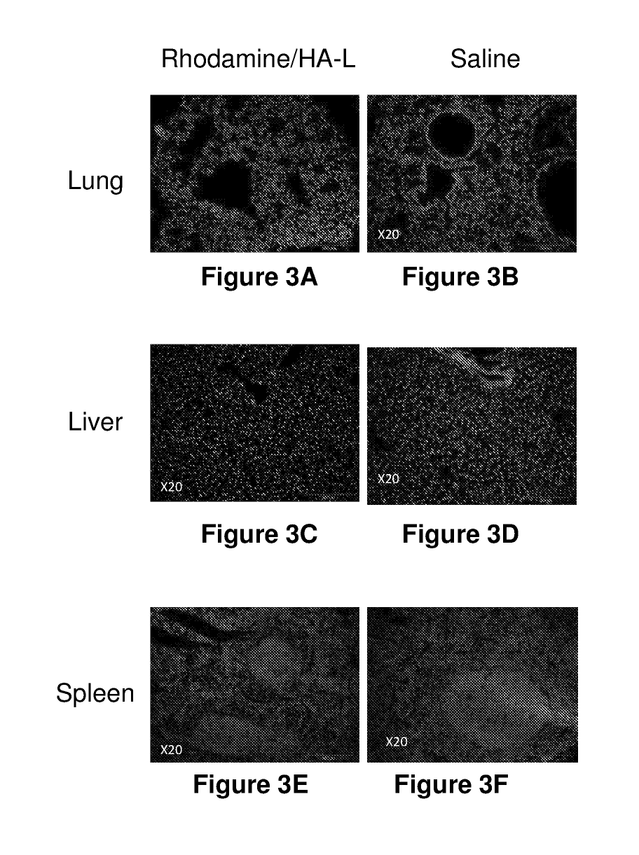

The second part of the targeting, namely no liposome accumulation other than in the MI heart was also met as seen in FIG. 2B and FIGS. 3A to 3F. As can be seen in these Figures, no accumulation of HA-L was observed in healthy control hearts, namely that did not undergo MI (FIG. 2B) as well as in other organs, aside of heart, obtained from mice undergoing MI (FIGS. 3A to 3F).

Thus, these results show that in vivo targeting of HA-L to infarcted heart of mice was successful.

### Fluorescently-Tagged HA-L in Atherosclerotic Mice

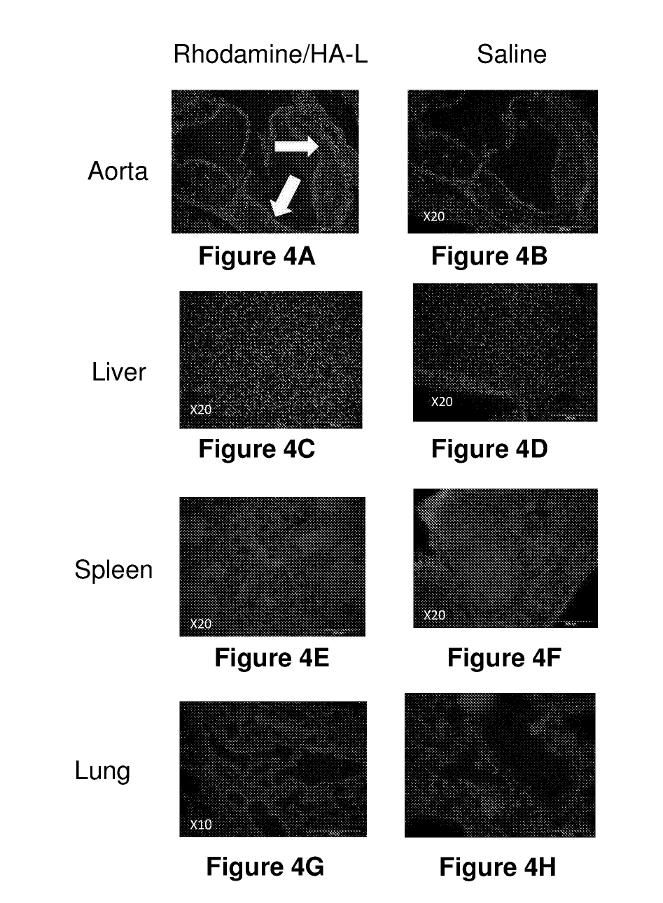

HA-L were injected i.v. (tail vein) to Apolipoprotein E (ApoE) KO mice. Six hours after injection, mice were euthanized and the inflamed aorta and other selected organs were cryo-sectioned to detect liposomes. As shown in FIGS. 4A to 4H, active targeting was observed also in this case, namely—high liposome accumulation in the aorta plaque (shown by the arrow) and no accumulation in other organs, such as liver and kidneys.

Thus active in vivo targeting to the inflamed atherosclerotic aorta in mice was observed.

In Vivo Active Targeting to a MI Pig with IONP-Encapsulating HA-L

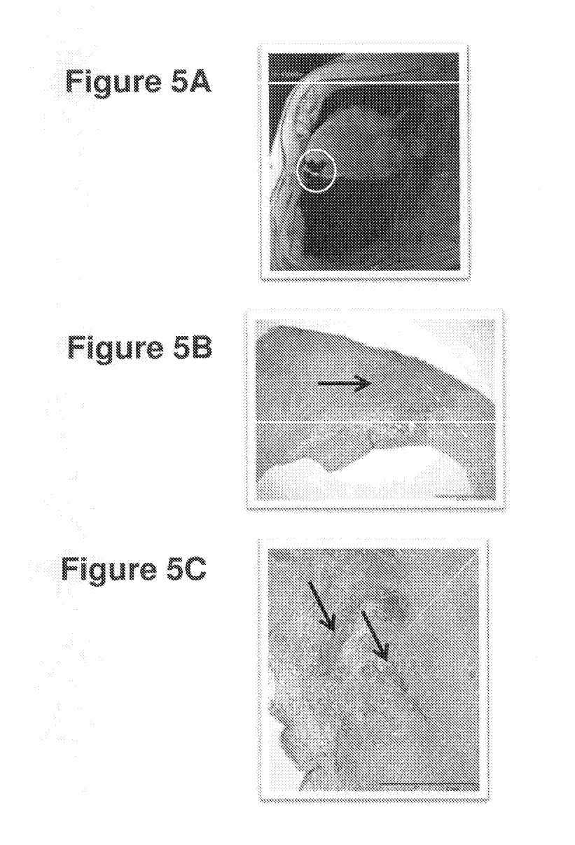

For the purpose of determining targeting and accumulation of the HA-L to the heart of infracted pigs, HA-L encapsulating iron oxide nanoparticles (IONP) were used for detection by MRI (IONP were used as contrast agent). In addition, the same liposomes were used for histology using iron staining. IONP-formulated HA-L liposomes (2 to 4 ml) were injected i.c. into the infarct artery or i.v. by a micro catheter, three days after MI induction (FIGS. 5A-5C). The liposome solution was injected manually at a pressure and rate similar to contrast media injection during coronary angiography.

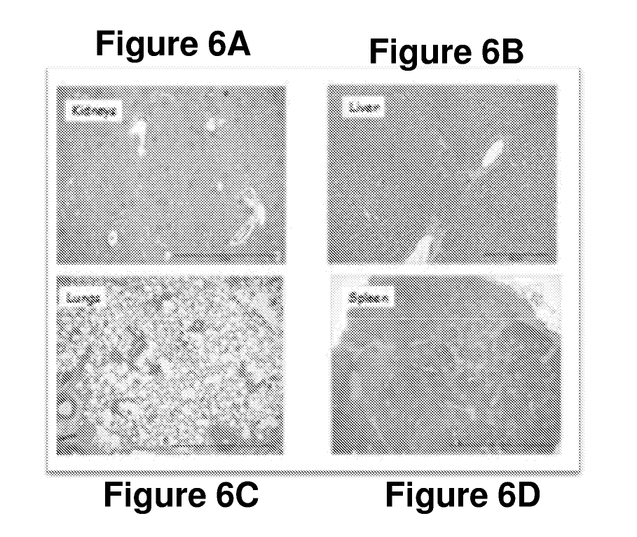

The data clearly shows accumulation of the IONP/HA-L in the infarcted heart using MRI (FIG. 5A, indicated by white circle) as well as in the histology analysis of the same zone shown in FIG. 4A (FIGS. 5B and 5C, the zone also being marked by arrows). FIG. 6A-6D shows histology of other organs showing no iron staining.

### Hemin Formulation in HA Targeted Liposomes

Hemin was encapsulated in liposomes defines as regular multilamellar vesicles (RL-MLV) and in hyaluronan targeted multilamellar veslicles (HA-L). The optimal lipid composition for both was phosphatidylcholine:cholesterol:phosphatidylethanol amine at a mole ratio of 55:40:5. The encapsulation efficiencies were very high, 92(±10)% and 91(±6)% for hemin in RL-MLV and in HA-L respectively.

### In Vitro Viability Assay

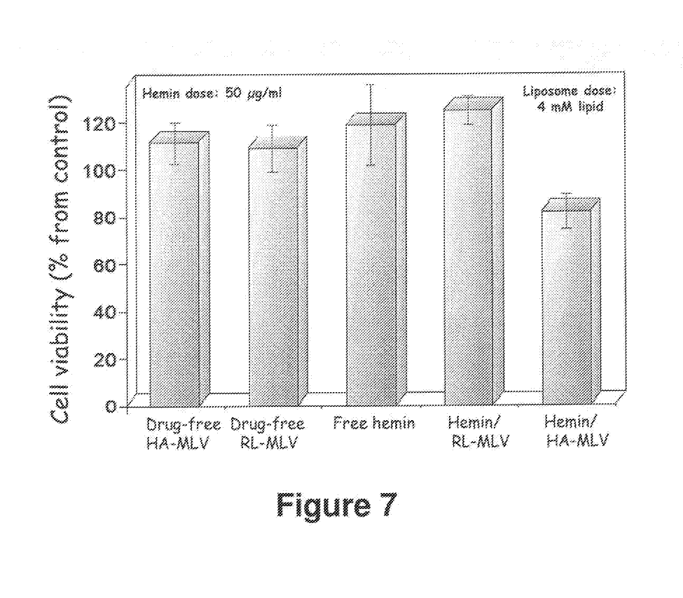

Cell viability was determined using XTT method. The results of FIG. 7 show that treatment of RAW274.6 cells with free hemin, hemin-free liposomes (RL-MLV and HA-MLV) as well as hemin-encapsulating RL-MLV and HA-MLV did not significantly affected cell viability. These results indicate an in vitro safety profile for all tested treatments and specifically to hemin encapsulated in liposomes, either regular liposomes or HA-liposomes. In addition, cell viability was also determined by the SRB method, which is independent of mitochondrial enzymes (results not shown). Both methods gave similar results.

### Example 1: Liposomal Hemin: In Vitro Evaluation of Anti-Inflammatory Activity

The anti-inflammatory activity of hemin was evaluated in vitro, in the established cell line of mouse peritoneal macrophages RAW264.7. Anti-inflammatory treatment was measured by reduction in secretion of the pro-inflammatory cytokine TNF-α, in LPS-activated macrophages. The results obtained for the hemin dose of 50 μg/ml are shown in FIG. 1C.

Free hemin and drug-free RL-MLV had no effect on TNF-α secretion. The hemin-encapsulating RL-MLV had a minor effect, as did drug-free HA-L. The latter result fits with knowledge in the field that hyaluronan itself has a minor anti-inflammatory effect that is not sufficient and therefore needs the addition of anti-inflammatory drugs. In contrast, the hemin-encapsulating HA-L had a highly significant effect, almost abolishing the TNF-α secretion. The hemin-encapsulating HA-L were thus determined to be a unique formulation that shows, in vitro, high efficacy as an anti-inflammatory treatment.

### Hemin/HA-L Increased M2/M1 Proportion.

Following hemin/HA-L (2 mg hemin/kg body and 50 mg lipid/kg body) injection into mice peritoneum (IP), macrophages from the abdominal cavity were isolated and analyzed by flow cytometry.

No significant difference was shown in number of macrophages (F4/80+ cells; FIG. 8A) among the different treated mice groups.

However, as shown in FIGS. 8B to 8D, M1 (pro-inflammatory) and M2 (anti-inflammatory) macrophage profile varied among the different treated mice groups. while free hemin increased both M1 (80.8.3±1, FIG. 8B) and M2 (6.4±1.5%, FIG. 8C), the empty liposomes decreased them (M1: 42±10% FIG. 8B and M2: 0.5±0.2%; FIG. 8C).

Hemin/HA-MLV also showed a decrease in M2 (1.1±0.5%) and even a greater decrease in M1 cells (10.3±1.7%), compared with saline (M1: 69±%1.4%; FIG. 8B and M2: 4.3±1.9%; FIG. 8C). In addition, M2/M1 was the highest among hemin/HAMLV treated mice (0.1±0.03), compared with free hemin (0.08±0.03), empty liposomes (0.011±0.01) and saline (0.06±0.03) (FIG. 8D).

In addition, no difference was observed among the F4/80+ cardiac macrophage accumulation following different treatments (FIG. 8E) while the Hemin/HA-MLV therapy increased M2/M1 ratio also in infarcted heart (FIG. 8F) as compared to the saline injection, free hemin or empty HA-MLV treatment.

### Hemin/HA-L (2 mg Hemin/Kg Body and 50 mg Lipid/Kg Body) Attenuated Left Ventricle (LV) Remodeling and Improved Heart Function

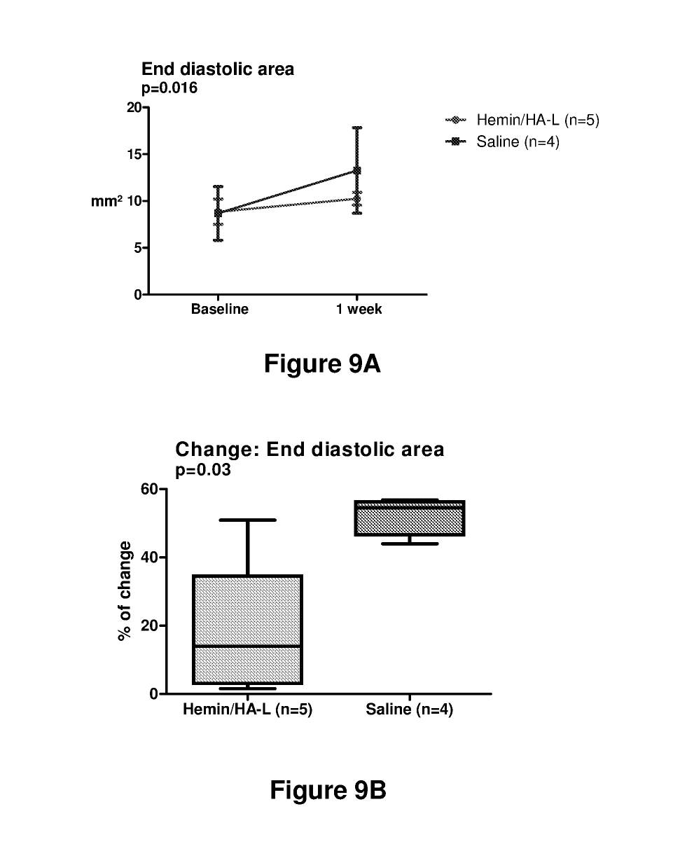

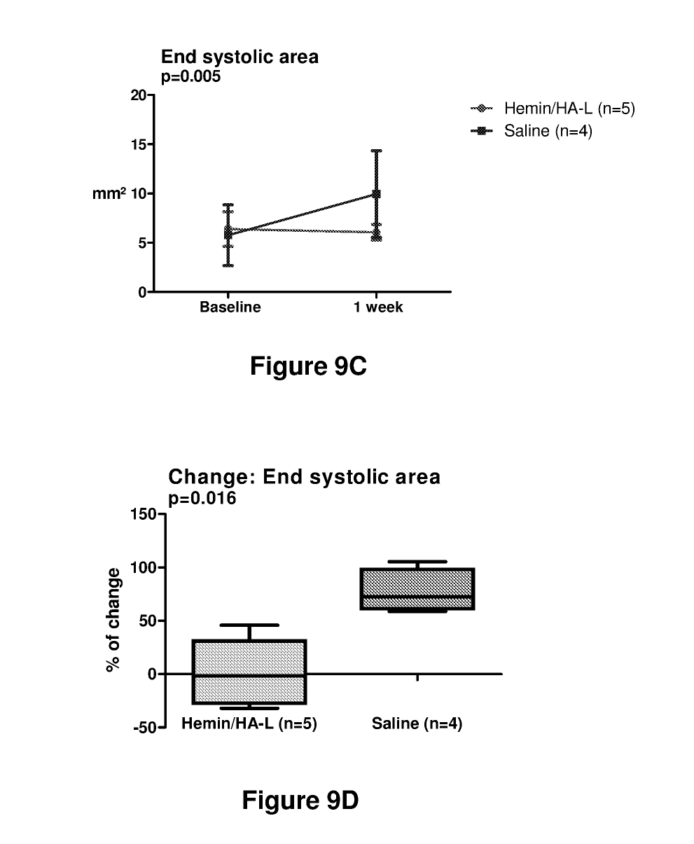

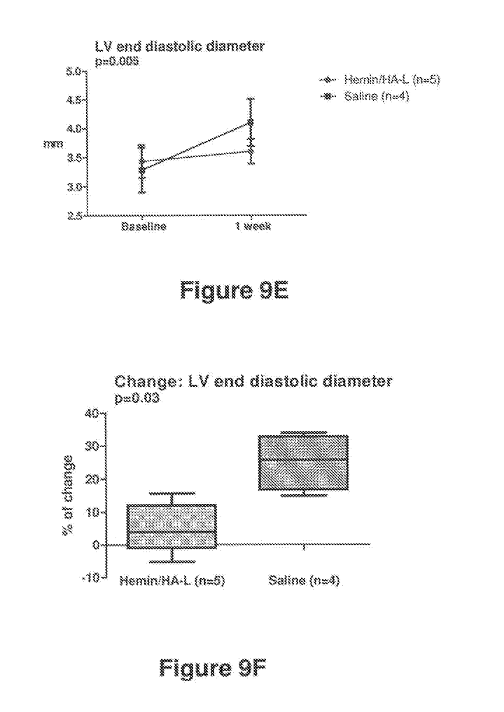

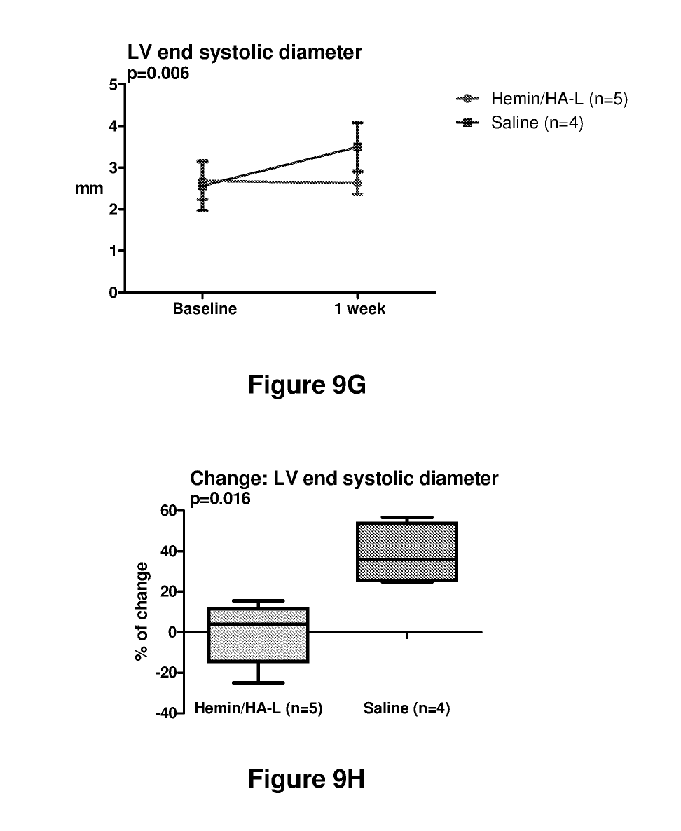

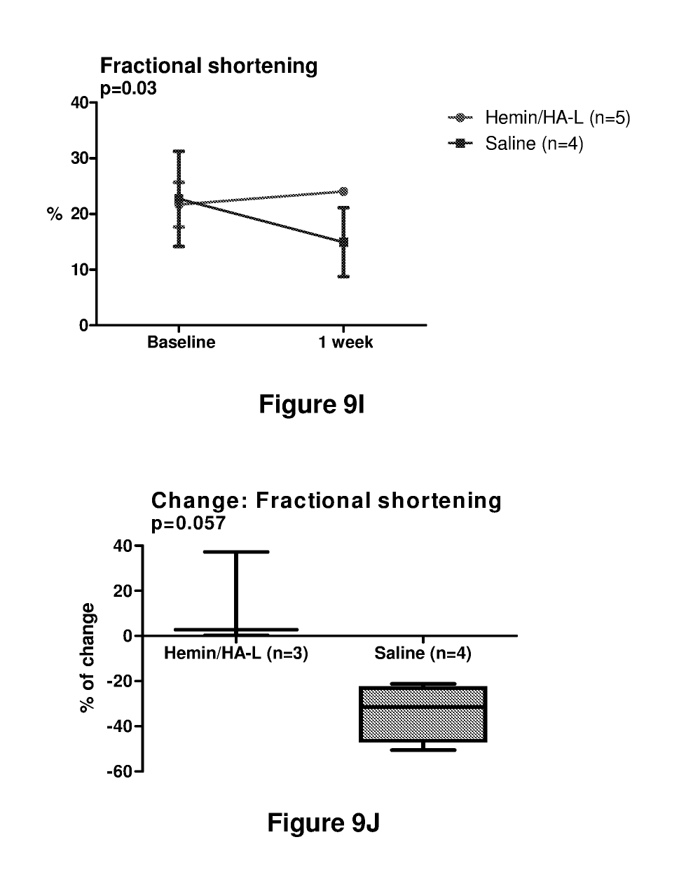

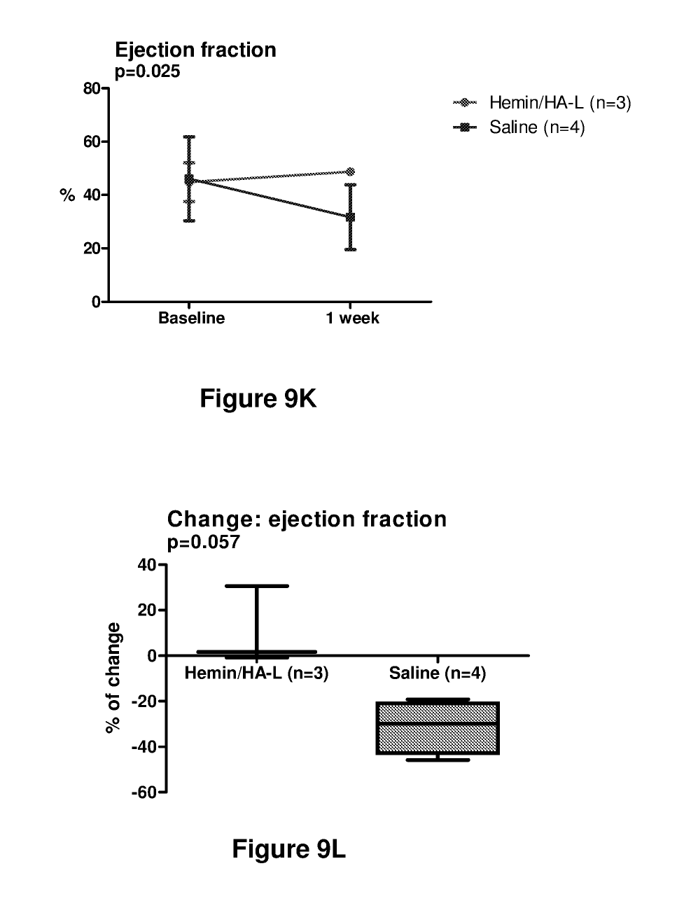

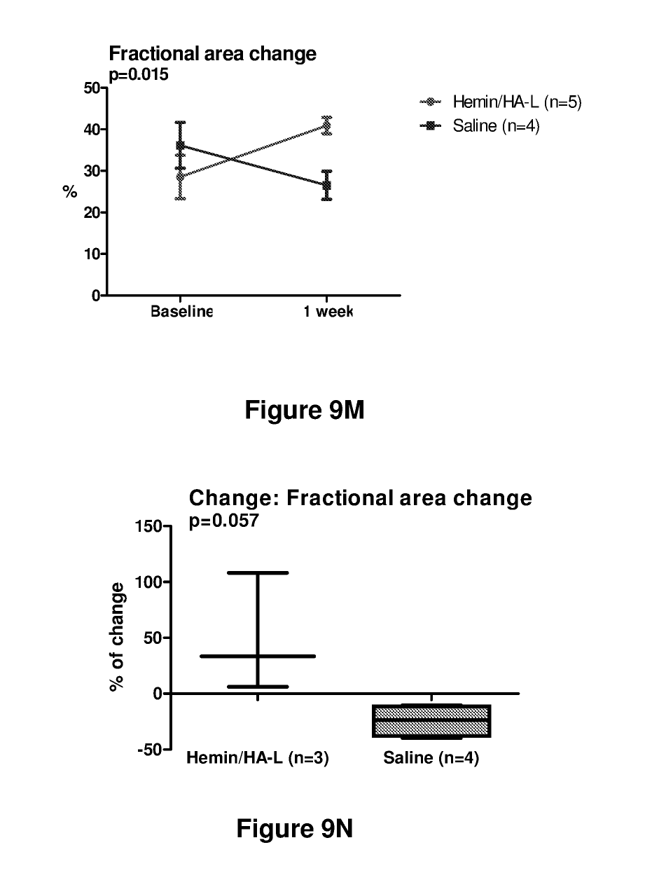

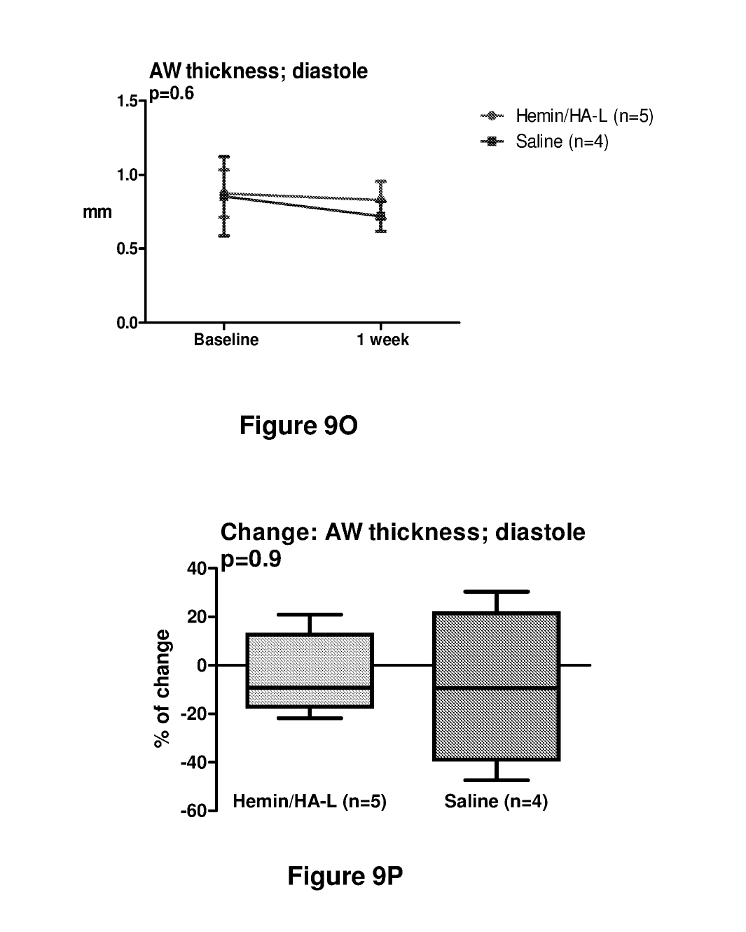

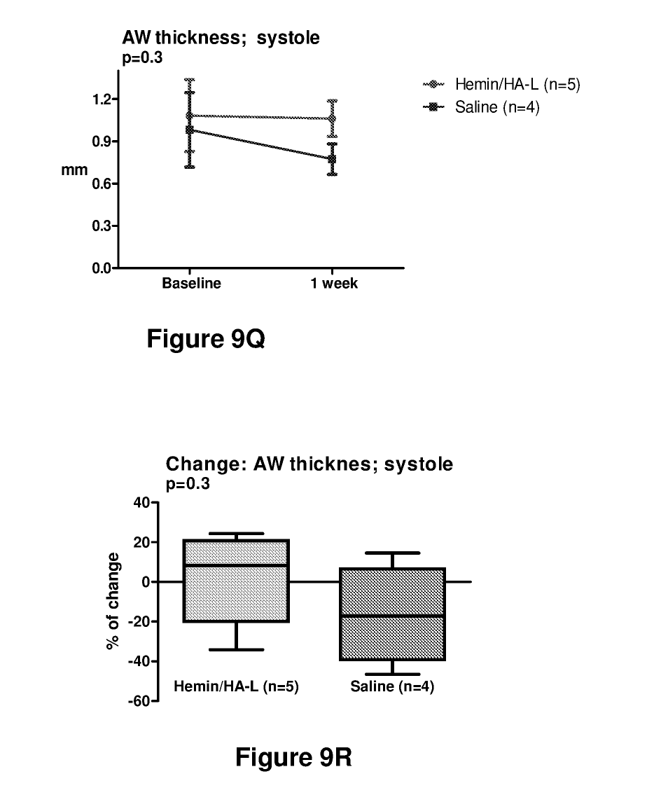

Hemin/HA-L abolished the typical deleterious effects from MI, one week after treatment (Table 1, FIG. 9). LV diastolic and systolic areas, as well as diameters, were significantly smaller after Hemin/HA-L therapy, compared with control (FIGS. 9A to 9H).

Moreover, LV function, indicated by fractional shortening, ejection fraction, and fractional area change, was improved after Hemin/HA-L therapy (FIGS. 9I to 9N), compared with saline treated mice. Finally Hemin/HA-L preserved post-MI systolic wall thickening, compared with the control group (FIGS. 9O to 9R).

TABLE 1
Hemin/HA-L (2 mg hemin/kg body and 50 mg lipid/kg body) attenuated
LV remodeling and improved heart function by echocardiography.

| | | Hemin/HA-L (n = 5) | Saline (n = 4) | P (repeated measured ANOVA) |
|---|---|---|---|---|
| End diastolic area (mm²) | Day 1 after MI | 8.84 ± 0.6 | 8.6 ± 1.4 | p interaction = 0.016 |
| | Day 7 after MI | 10.2 ± 0.2 | 13.2 ± 2.3 | p treatment effect = 0.4 |
| | | | | p time effect-0.0006 |
| End systolic area (mm²) | Day 1 after MI | 6.4 ± 0.8 | 5.7 ± 1.5 | p interaction = 0.005 |
| | Day 7 after MI | 6 ± 0.3 | 9.9 ± 2.2 | p treatment effect = 0.37 |
| | | | | p time effect = 0.011 |
| LV end diasolic diameter (mm) | Day 1 after MI | 3.4 ± 0.12 | 3.2 ± 0.2 | p interaction = 0.005 |
| | Day 7 after MI | 3.6 ± 0.09 | 4 ± 0.2 | p treatment effect = 0.4 |
| | | | | p time effect = 0.0005 |
| LV end systolic diameter (mm) | Day 1 after MI | 2.7 ± 0.2 | 2.5 ± 0.3 | p interaction = 0.005 |
| | Day 7 after MI | 2.6 ± 0.12 | 3.5 ± 0.3 | p treatment effect = 0.4 |
| | | | | p time effect = 0.0005 |
| Fractional shortening (%) | Day 1 after MI | 21.6 ± 2.3 | 22.7 ± 4.2 | p interaction = 0.03 |
| | Day 7 after MI | 24 ± 0.3 | 15 ± 3 | p treatment effect = 0.4 |
| | | | | p time effect = 0.17 |
| Ejection fraction (%) | Day 1 after MI | 44.8 ± 4.2 | 46 ± 7.8 | p interaction = 0.025 |
| | Day 7 after MI | 48.7 ± 0.5 | 31 ± 6 | p treatment effect = 0.4 |
| | | | | p time effect = 0.13 |
| Fractional area change (%) | Day 1 after MI | 28.5 ± 5.2 | 36.1 ± 5.5 | p interaction = 0.015 |
| | Day 7 after MI | 41 ± 1.9 | 26.5 ± 3.4 | p treatment effect = 0.5 |
| | | | | p time effect = 0.7 |
| Anterior wall thickness; d (mm) | Day 1 after MI | 0.87 ± 0.07 | 0.85 ± 0.1 | p interaction = 0.06 |
| | Day 7 after MI | 0.83 ± 0.05 | 0.72 ± 0.005 | p treatment effect = 0.4 |
| | | | | p time effect = 0.3 |
| Anterior wall thickness; s (mm) | Day 1 after MI | 1.08 ± 0.1 | 0.98 ± 0.1 | p interaction = 0.36 |
| | Day 7 after MI | 1.06 ± 0.05 | 0.77 ± 0.05 | p treatment effect = 0.08 |
| | | | | p time effect = 0.27 |

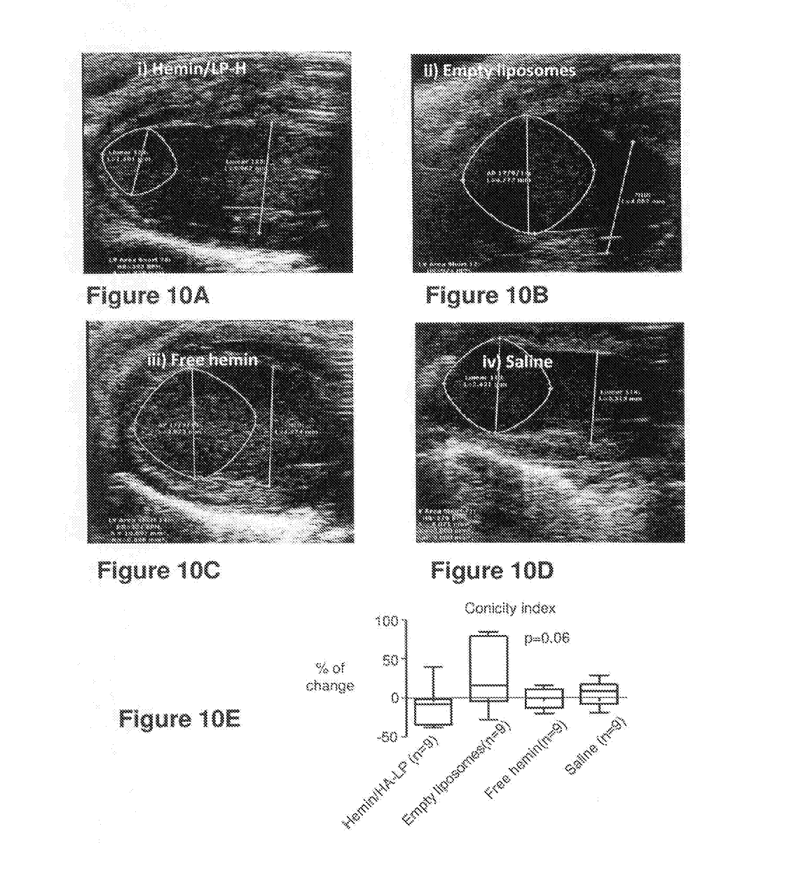

In addition, to describe structural changes occurring after MI, conicity and sphericity indexes among study groups were compared (FIGS. 10A-10E). After LAD occlusion in mice, the LV started to alter towards a balloon shape. Remodeling was initially regional, starting from the infarcted apex and spreading toward the anterior mid with time. To determine treatment effect on infarct expansion (thinning and dilatation), conicity index at 7 days after MI was compared by calculating the ratio of apical diameter to mid diameter (FIG. 10A-10D). Remarkably, Hemin/HA-LP lowered conicity index compared with other groups, indicating smaller apical infarcts and dilatation (FIG. 10E).

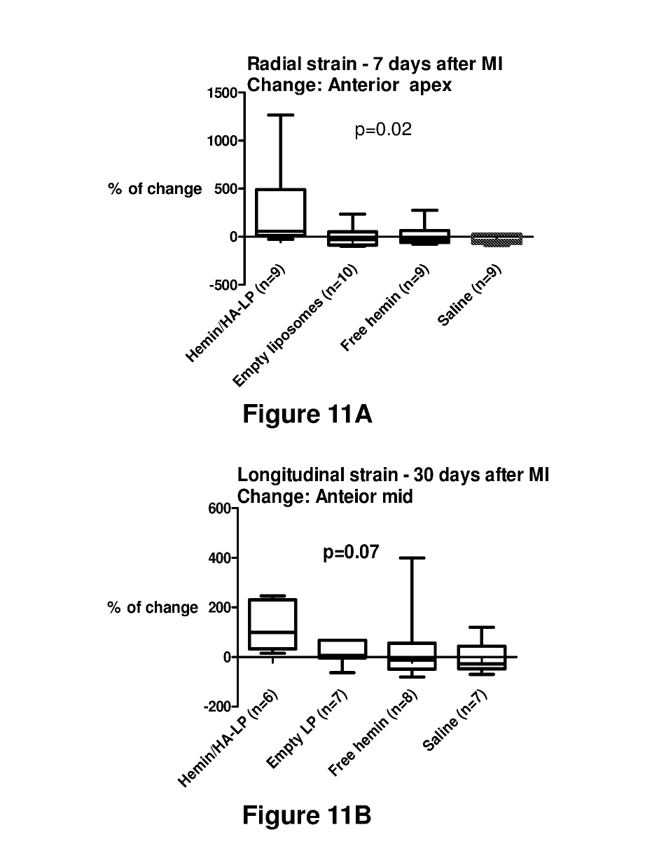

In order to confirm improvement in regional myocardial function after MI by LV speckle-tracking based strain analysis, a more sensitive method for assessing global and region-specific myocardial contractility was conducted, the results of which are shown in FIG. 11A-11B. Significantly, hemin/HA-LP treatment improved radial apical strain, seven days after MI (FIG. 11A; p=0.02). Furthermore, hemin/HA-LP treatment also improved longitudinal anterior mid strain 30 days after MI, compared with other study groups (FIG. 11B).

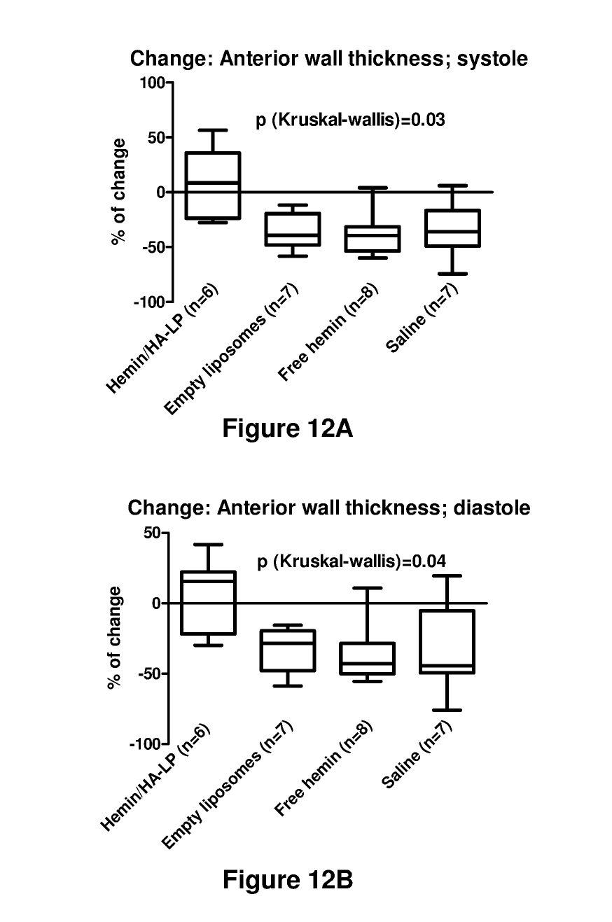

Further, to determine hemin/HA-LP effect (after 30 days) on LV wall thickness, anterior wall thickness was measured by echocardiography at systole (FIG. 12A) and diastole (FIG. 12B). Significantly, while control groups developed LV anterior wall thinning after MI, hemin/HA-LP-treatment preserved infarct-related anterior wall thickness. Overall, the successful prevention of infarct thinning and reduced infarct strain (by Laplace low), is a possible explanation for attenuation of remodeling and LV dilatation in treated hearts.

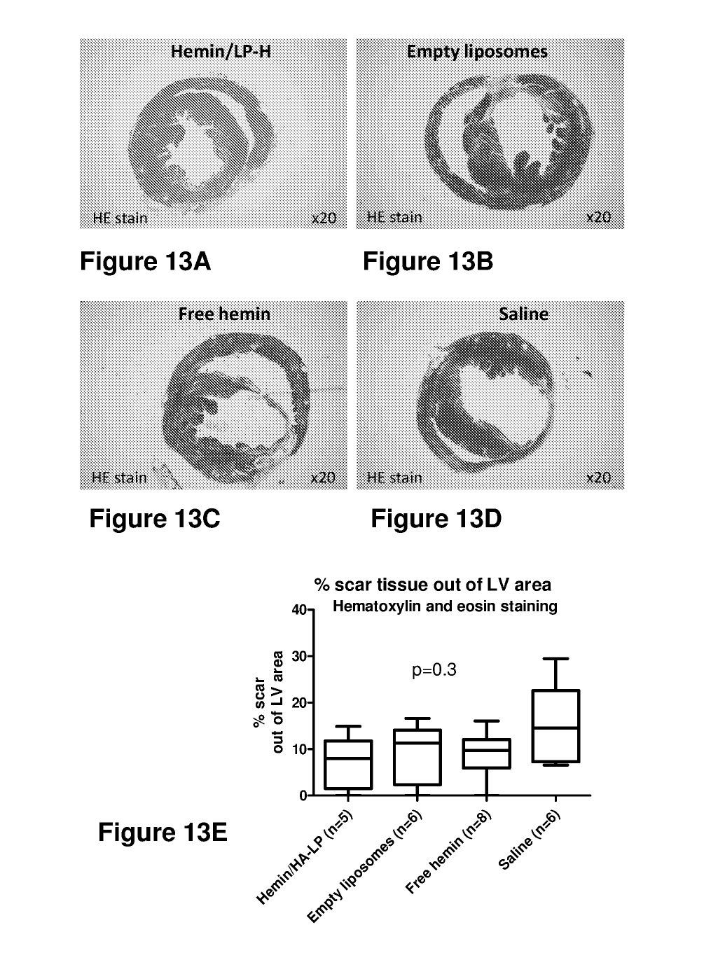

The imaging results of treatment with Hemin/HA-L (FIG. 13A), free hemin (FIG. 13B), empty liposomes (FIG. 13C) and saline (FIG. 13D) were confirmed by histological analysis, 30 days after MI. Specifically, hematoxylin and eosin staining revealed that hemin/HA-L reduced infarct size by 56%, compared with saline (FIG. 13E). Noticeably, empty liposomes and free hemin treatments were also associated with a decreased scar tissue (by 40% and 43%), compared with saline (FIG. 13E). Overall, the presented findings indicate that hemin/HA-L is a promising agent to modulate and resolve inflammation following MI by enhancing healing and repair processes.

### Example 2: Hemin Encapsulating HA-Liposomes (Hemin/HA-L) and Viability of Macrophages, Cancer and Non-Cancer Cell Lines

### Effect on Different Cell Types

For evaluating the effect of Hemin/HA-L on cancer cells, the liposomes prepared as described above were used. The only difference was in the amount of dry liposome forming lipid powder used in the “cancer aspect”. Specifically, in the following anti-cancer aspect, for rehydration of the hemin, higher amounts of the powder were used to reach a liposome concentration of 10-20 mM lipid.

The impact of hemin/HA-L at high liposome concentrations on cell viability was tested on five cell lines and compared to the same dose of free hemin, as well as to the same liposomal dose of drug-free HA-L.

Three of the cell lines originated from human cancers: MDA-MB-231 and MCF-7 from breast cancer and NCI/ADR-Res (abbreviated to NAR) from ovarian cancer. The RAW264.7 cell line is from mouse peritoneal macrophages, and the COS-7 cell line is noncancerous. The macrophages and the cancer cell lines all harbor the CD44 receptors on their cell membranes whereas the COS-7 line does not express CD44 receptors. MCF-7 is known to be sensitive to chemotherapeutic drugs, whereas NAR is known to be highly resistant to chemotherapeutic drugs, in the multi-drug resistant mechanism operating by the extrusion pumps of the ABC superfamily of transporters.

The Results of treating the five cell lines with hemin/HA-L, hemin, drug-free HA-L or control (i.e. untreated) for 24 hours, are shown in Tables 2 and 3, for hemin doses (free or liposomal) of 25 μg/ml and 50 μg/ml, respectively. Cell viability was evaluated by two independent methods: XTT and SRB.

Table 2 shows the effect of equi-doses of free and of liposomal hemin on cell viability as function of liposome (HA-L) doses and cell type, for the hemin dose of 25 μg/ml.

TABLE 2

| | Cell Viability (%)* | | | |
|---|---|---|---|---|
| | Drug-free | | | |
| | HA-L | | Hemin/HA-L | |
| Cell line | 10 mM lipid | Free hemin | 2 mM lipid | 10 mM lipid |
| COS-7 | 58(±3) | 99(±3) | | 83(±1) |
| RAW264.7 | 86(±12) | | 90(±7) | 6(±1) |
| MCF-7 | 112(±8) | 104(±4) | 91(±6) | 5(±0.4) |
| MDA-MB-231 | 125(±33) | 92(±13) | 101(±11) | 20(±1) |
| NCI/ADR-Res | 94(±8) | 120(±7) | 62(±8) | 15(±2) |

*Each value is an average of at least 6 wells, and the numbers in parenthesis are the standard deviations.

Table 3 shows the effects of equi-doses of free and of liposomal hemin on cell viability as function of liposome (HA-L) doses and cell type, for the hemin dose of 50 μg/ml.

TABLE 3

| | Cell Viability (%)* | | | |
|---|---|---|---|---|
| | HA-L | | Hemin/HA-L | |
| Cell line | 21 mM lipid | Free hemin | 4 mM lipid | 21 mM lipid |
| COS-7 | 62(±1) | 91(±3) | | 78(±2) |
| RAW264.7 | 111(±9) | 119(±17) | 82(±7) | 7(±1) |
| MCF-7 | 109(±13) | 108(±5) | 60(±8) | 5(±0.4) |
| MDA-MB-231 | 113(±24) | 91(±8) | 78(±8) | 25(±1) |
| NCI/ADR-Res | 105(±6) | 110(±6) | 23(±7) | 19(±3) |

*Each value is an average of at least 6 wells, and the numbers in parenthesis are the standard deviations.

The results presented in Table 2 and in Table 3 show that for the COS-7 cells, treatment with free or liposomal hemin, at both hemin doses and at both low and high liposome doses, did not generate any significant loss in cell viability. These cells do not express the CD44 receptors and, thus, as expected, do not show any significant difference between free and liposomal hemin (at both hemin doses).

As also seen in Tables 2 and 3, viability of the macrophages and the cancer cell lines was not harmed by the free hemin (both doses). The situation changes with the liposomal hemin. At the lowest liposome dose (2 mM lipid, Table 2) and at the low hemin dose (i.e. 25 μg/ml) the viability of the macrophages and the two breast cancer cell lines remains unaffected, while that of the ovarian cell line already shows a modest drop. At the same hemin dose but at the higher liposome dose (10 mM lipid, Table 2) the viability of the macrophages and all three cancer cell lines drops significantly. Similar phenomena, but with some dose-dependent quantitative differences, were also observed with the higher hemin dose (50 μg/ml, Table 3). At the lower liposome dose (4 mM lipid, Table 3), viability of the macrophages and the two breast cancer cell lines drops mildly, and that of the ovarian cell line already shows significant drop. At the high liposome dose (21 mM, Table 3), there is a significant drop is the viability of the macrophages and all three cancer cell lines. As also seen in Tables 2 and 3, the viability of the macrophages and the cancer cell lines remains unaffected when treated with drug-free HA-L, indicating that the liposomes themselves even at the high doses are not toxic to the cells.

In summary, for the same hemin dose and at high liposome doses, the liposomal hemin was quite cytotoxic to macrophages and the cancer cell lines originating from human tumors. There was some cell-line specificity in terms of the levels of cell death, which is something to be expected.

### Effect of Lipid Concentration on HO-1 Level and Cell Viability

The inventors have realized that the effect of hemin/HA-L on cell viability is by (1) the level of intracellular HO-1 induced by hemin, which is significantly higher with hemin/HA-L than with an equi-dose of free hemin and/or (2) Cell death only due to hemin/HA-L, cell viability intact with an equi-dose of free hemin.

Specifically, without being bound by theory it is postulated that as long as entry of hemin into a macrophage does not harm cell viability, an anti-inflammatory of the macrophage will come into effect. To test this understanding the anti-inflammatory activity (reducing LPS-induced TNF-α secretion) and viability of RAW264.7 cells as a function of hemin formulation (free or encapsulated) was determined. FIG. 1C, shows that the anti-inflammatory (immunomodulatory) activity of the same hemin dose depends on its formulation—at the dose given (50 μg/ml hemin), free hemin did not generate any anti-inflammatory (immunomodulatory) activity while that of the liposomal hemin was quite substantial. the anti-inflammatory activity as detailed under Example one). FIG. 7 is a bar graph showing the cell viability for the same experiment illustrated in FIG. 1C, confirming that at the low liposome dose used, there was no significant loss in cell viability. (each bar being an average of 6-10 wells, Statistical significance of cell viability was evaluated for free hemin and for hemin/HA-L, each vs. drug-free HA. *p<0.05, ***p<0.001).

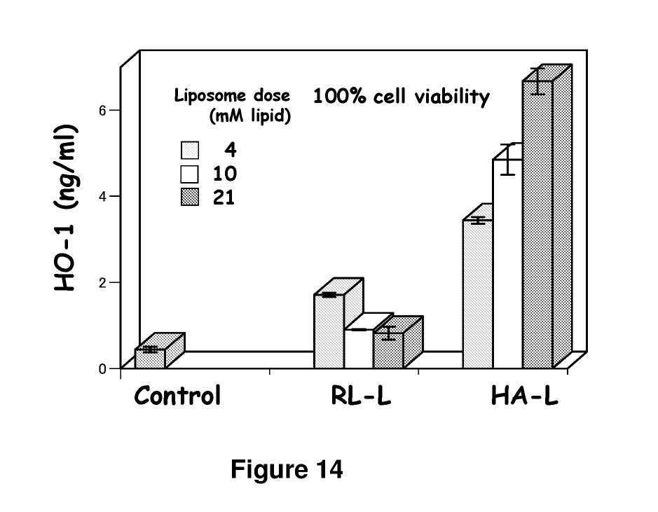

The inventors have further found that there is a difference in effect depending on lipid concentration. To this end, NCI/ADR-Res (NAR) cells were first tested with drug-free regular liposomes and drug-free HA-L, at three liposome doses—4 mM, 10 mM, and 21 mM—lipid, with untreated cells serving as controls. The experiments were performed in 24-well plates, treatment was for 24 hours, and upon termination the active intracellular HO-1 was determined using a commercial kit and following the kit's instructions. Parallel wells were used to evaluate cell viability using the SRB method. The results are presented in FIG. 14 (average of 6 wells; Statistical significance was evaluated by student t test (two tail, unequal variance) for RL vs. control and for HA-L vs. control. *p<0.05, **p<0.003, ***p<0.0001).

As can be seen in FIG. 14 the basal level of HO-1 in NAR cells is quite low. Drug-free regular liposomes did have a small impact, but with dependence on the liposome doses. Cell viability was intact for both types of drug-free liposomes.

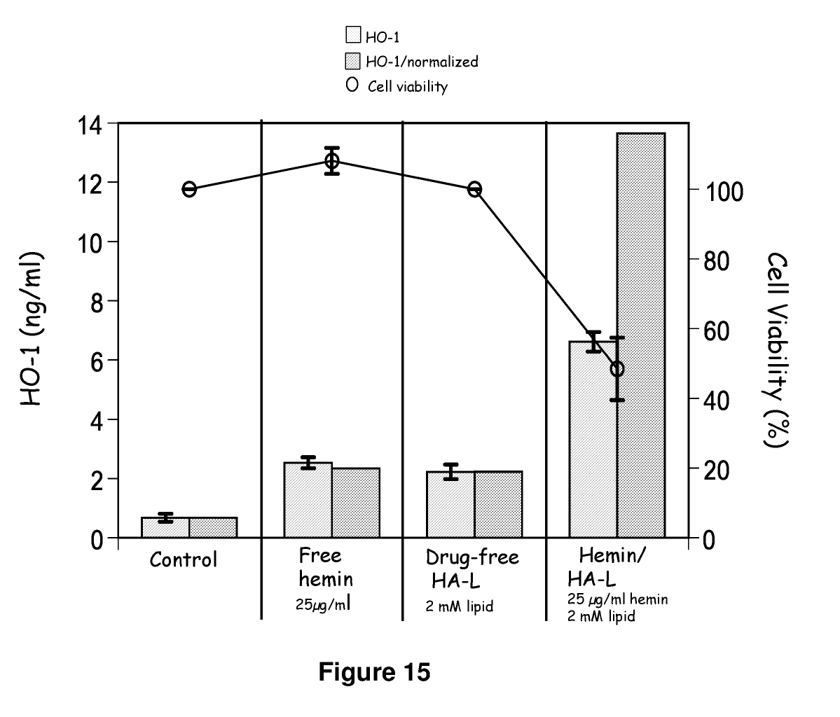

Next, the impact of free and liposomal hemin, together with several controls, on intracellular levels of active HO-1 and on cell viability for the same cell line (i.e. NCI/ADR-Res (NAR) was tested. The protocol was the same as described above for the drug-free liposomes. As can be seen in FIG. 15 (control and drug free HA-L are as in FIG. 14), statistical significance was evaluated by student t test (two tail, unequal variance), for HO-1 activity free hemin, drug-free HA-L and hemin/HA-L were compared, each, vs., control. For viability, hemin/HA-L was compared vs. control ***p<0.0003), free hemin at the dose of 25 μg/ml induced a small, but significant increase in HO-1 which was of a level similar to drug-free HA-L at 2 mM lipid. In both cases, as also shown in FIG. 15, cell viability (line crossing open circles) was intact. Administering the same hemin dose—but encapsulated in the same HA-L dose (i.e., 25 μg/ml hemin and 2 mM lipid)—generated a significant increase in HO-1, with a concomitant 50% drop in viable cells. Normalizing all HO-1 concentrations in FIG. 15, to 100% viable cells, shows that the impact of hemin/HA-L on the increase in HO-1 was more than 10 fold over the basal level.

In summary, the data in FIGS. 14 and 15 show that Hemin/HA-L increased the intracellular levels active HO-1 significantly higher than equi-doses of free hemin, and brought about cell death. Interestingly, as shown in FIG. 12, drug-free HA-L had a modest effect of increasing the HO-1 but without bringing about cell death. This is understandable, since the drug-free HA-L only increased the enzyme—without supplying the substrate (i.e., hemin). This finding fits with other indirect indications that hyaluronan itself has a mild anti-inflammatory activity.

### In Vivo Studies in a Mouse Tumor Model, Pursuing the Anti-Cancer Activity of Hemin/HA-L

Based on the in vitro studies, treatment of NAR xenografts by hemin/HA-L at high lipid concentrations (hereinafter hemin/HA-L^{hi}) was conducted to test whether it slows down tumor progression and whether such a treatment is safe.

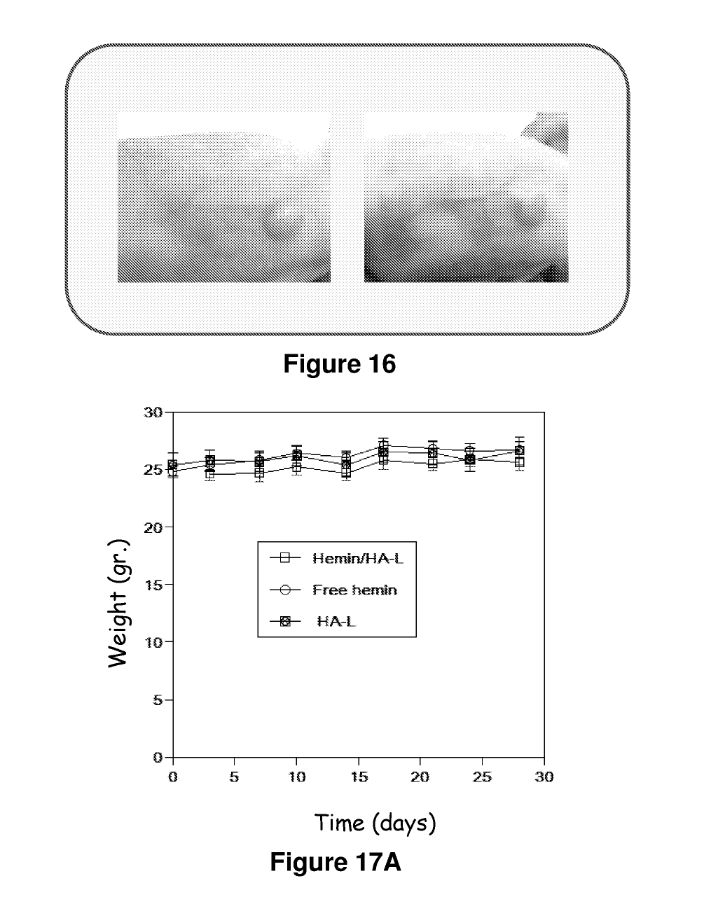

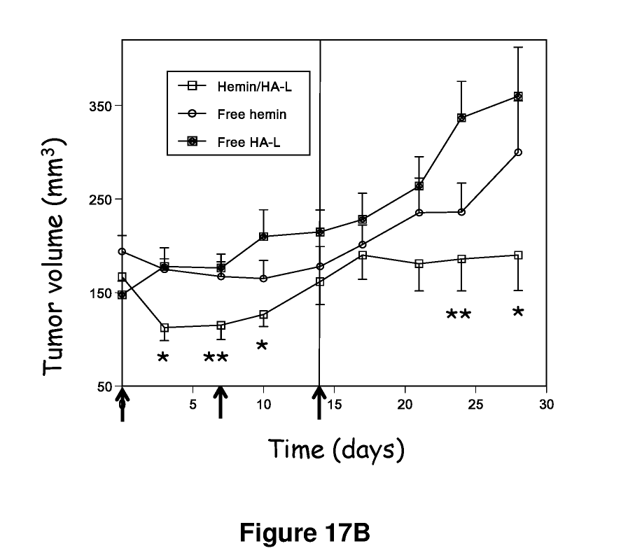

The experimental details and design were as follows: Hsd:Athymic Nude Foxn1nu female mice (8 weeks old, 25 gr.) were inoculated with NAR cells (6.5*10⁶ cells/100 μl HBSS), to the flank. Treatment was initiated at tumor volumes of 150-180 mm³. There were three treatment groups and in all three the solvent was HBSS: (1) mice injected with drug-free HA-L^{hi} at the dose of 125 mpk lipid. (2) mice injected with free hemin at the dose of 2 mpk and (3) mice injected with hemin/HA-L^{hi} at the respective doses of 2 mpk (hemin) and 125 mpk (lipid). The injections were to the tail vein, and the injection volume was 200 μl. Each mice was given 3 injections spaced a week apart. Follow up was by animal weight and tumor volume (measured by caliper), up to two weeks after the last injection. The results are shown in FIG. 16 and FIG. 17A-17B.

FIG. 16 provides images of the tumor area from two different mice. FIG. 17A provides weight changed due to treatment and in particular shows that there were no drops in mice weight, in either of the groups, indicating treatment safety, which also fits with the fact that all animals survived during the experimental run. As can be seen in the images of FIG. 16, the tumors were well vascularized.

FIG. 17B shows the change in tumor volume with time and treatment with each point being an average of all animals in the group (error bars are the SEM, Statistical significance was evaluated by student t (unequal variance, two tail) for hemin/HA-L vs. drug-free hemin. *p<0.03, **p<0.014). The arrows indicate the days of injection. Filled squares represent animals receiving drug-free HA-L (n=7) while circles represent animals receiving free hemin (n=8) and open squares represent animals receiving hemin/HA-L. The animals were treated with doses of 2 mpk (mpk=mg/kg body) hemin and 125 mpk lipid (liposomes).

Specifically, shown in FIG. 17B, the tumor volumes of the control mice—receiving drug-free HA-L^{hi}—increased exponentially from the first injection day and on. The mice receiving free hemin showed a slight decrease in tumor volume during the treatment period which was not significantly different from the control mice, and an exponential increase in tumor volume once treatment was ended. The mice receiving the test system—hemin/HA-L^{hi}—showed a significant decrease in tumor volumes during the treatment period which persisted, presenting indications of tumor arrest, even two weeks after the last injection. The inventors have attribute the latter to the slow-release nature of the targeted HA-L that are bound to the cells as depots with the potential to continue supplying drug to the cells after dosing has terminated.

Taking together the data of FIG. 16 and FIGS. 17A-17B, it shows that hemin/HA-L^{hi} has, indeed, a tumor treatment ability which is both efficacious and safe when used at the appropriate concentration of lipid. The NAR cell line is known to be, in vitro and in vivo, highly resistant to conventional chemotherapy, by the mechanism of the MDR extrusion pumps.

Further, the data presented herein support the conclusion that there is presented herein a novel safe and efficacious tumor treatment that is applicable for drug-sensitive cancer cells (such as MCF-7, see Tables 2 and 3), drug-resistant cancer cells (Tables 2 and 3, and related Figures), as well as tumor-associated macrophages (Tables 2 and 3).

1.-44. (canceled)

45. A liposome comprising a lipid membrane enclosing an intraliposomal compartment, said liposome: (i) encapsulates in the intraliposomal compartment an iron complex comprising an iron moiety and a macromolecule; and (ii) has associated to its membrane a targeting moiety, the targeting moiety being exposed at the liposome's outer surface.

46. The liposome of claim 45, wherein said macromolecule is porphine or a macromolecule having a porphine backbone.

47. The liposome of claim 46, wherein the macromolecule is selected from the group consisting of porphine, porphyrin and protoporphyrin IX.

48. The liposome of claim 45, wherein said targeting moiety is hyaluronan (HA).

49. The liposome of claim 45, wherein said lipid membrane comprises up to 40% w/w sterol.

50. The liposome of claim 45, wherein said lipid membrane comprising phosphatidylcholine, cholesterol and phosphatidylethanol amine.

51. The liposome of claim 50, comprising said phosphatidylcholine, cholesterol and phosphatidylethanol at a mole ratio of 55:40:5.

52. The liposome of claim 45, comprising a molar ratio between the lipids of the lipid membrane and the macromolecule is in the range of 5 to 120 or in the range of 120 to 1,400.

53. A liposome comprising a lipid membrane enclosing an intraliposomal compartment, said liposome: (i) encapsulates in the intraliposomal compartment hemin; and (ii) has bound to its membrane hyaluronic acid (HA), the HA being exposed at the liposome's outer surface.

54. A therapeutic method comprising administering to a subject in need of treatment an amount of liposomes comprising a lipid membrane enclosing an intraliposomal compartment, said liposome: (i) encapsulates in the intraliposomal compartment an iron complex comprising an iron moiety and a macromolecule; and (ii) has associated to its membrane a targeting moiety, the targeting moiety being exposed at the liposome's outer surface.

55. The therapeutic method of claim 54, wherein said liposome is formulated with a physiologically acceptable carrier suitable for administration by injection or for oral administration.

56. The therapeutic method of claim 54, wherein said liposome comprises a molar ratio between lipids forming the lipid membrane and said macromolecule in the range of 5 to 120 or in the range of 120 to 1,400.

57. The therapeutic method of claim 56, wherein, when said molar ratio is in the range of 120 to 1,400, said method is for the treatment of cancer.

58. The therapeutic method of claim 56, wherein when said molar ratio is in the range of 5 to 120, said method is for the treatment of a non-infectious inflammatory condition.

59. The therapeutic method of claim 58, wherein the non-infectious inflammatory disorder is of the cardiovascular system.

60. The therapeutic method of claim 54, wherein said macromolecule is porphine or a macromolecule having a porphine backbone.

61. The therapeutic method of claim 54, wherein the macromolecule is selected from the group consisting of porphine, porphyrin and protoporphyrin IX.

62. The therapeutic method claim 54, wherein said lipid membrane comprising phosphatidylcholine, cholesterol and phosphatidylethanol amine.

63. The therapeutic method of claim 54, comprising said phosphatidylcholine, cholesterol and phosphatidylethanol at a mole ratio of 55:40:5.

64. A therapeutic method comprising administering to a subject in need of treatment an amount of liposomes comprising a lipid membrane enclosing an intraliposomal compartment, said liposome: (i) encapsulates in the intraliposomal compartment hemin; and (ii) has bound to its membrane hyaluronic acid (HA), the HA being exposed at the liposome's outer surface.
